(19) United States
(12) Patent Application Publication
(10) Pub. No.: US 2019/0159715 A1
(43) Pub. Date: May 30, 2019

(54) METHODS OF COGNITIVE FITNESS DETECTION AND TRAINING AND SYSTEMS FOR PRACTICING THE SAME

(71) Applicant: The Regents of the University of California, Oakland, CA (US)

(72) Inventors: Jyoti Mishra Ramanathan, San Francisco, CA (US); Adam Gazzaley, San Francisco, CA (US)

(21) Appl. No.: 16/321,393

(22) PCT Filed: Jul. 31, 2017
(86) PCT No.: PCT/US17/44670

§ 371 Date: Jan. 28, 2019

Related U.S. Application Data
(60) Provisional application No. 62/371,607, filed on Aug. 5, 2016.

(51) Int. Cl. A61B 5/00; A61B 5/0484

(52) U.S. Cl. CPC A61B 5/4088; A61B 5/04012; A61B 5/4064; A61B 5/0484

(57) ABSTRACT

Provided are methods of assessing and/or training cognitive fitness. Aspects of the instant methods generally relate to identifying and observing neural activity that underlies an event occurring in response to the stimulus or sequence of stimuli of a cognitive task performed by a subject. As such, the instant methods generally include presenting a cognitive task to a subject that includes a stimulus or sequence of stimuli, and monitoring the neural activity of the subject during performance of the cognitive task. Monitoring of such neural activity may be used, at least in part, to determine a neural performance level of a subject which may, in turn, be used in various ways including e.g., as an assessment of cognitive fitness, to tailor a subsequently presented cognitive task to train the cognitive fitness of the subject, etc. Systems and computer readable media for practicing the methods of the present disclosure are also provided.

### CROSS-REFERENCE TO RELATED APPLICATIONS

Pursuant to 35 U.S.C. § 119 (e), this application claims priority to the filing date of U.S. Provisional Patent Application Ser. No. 62/371,607, filed Aug. 5, 2016; the disclosure of which application is herein incorporated by reference in its entirety.

### INTRODUCTION

A general desire to improve cognitive function is widespread in the human population, in the young and old alike. Particularly in the United States, as the median age of the population increases, even clearly healthy consumers continue to look for ways to at least maintain cognitive function during aging and prevent cognitive decline.

Furthermore, cognitive impairment continues to be a significant health issue, both in the United Stated and globally. One relevant clinical example is mild cognitive impairment (MCI) which is classified as a slight but noticeable and measurable decline in cognitive abilities, including memory and thinking skills. A person with MCI is at an increased risk of developing Alzheimer's or another dementia.

The number of people living with dementia worldwide is currently estimated at 47.5 million and is projected to increase to 75.6 million by 2030. The number of cases of dementia is estimated to more than triple by 2050. Although dementia mainly affects older people, it is not a normal part of ageing. Dementia is a syndrome, usually of a chronic or progressive nature, caused by a variety of brain illnesses that affect memory, thinking, behavior and ability to perform everyday activities. Early diagnosis improves the quality of life of people with dementia and their families.

Cognitive impairments are not limited to the aged. For example, may young people, particularly adolescents, are known to suffer from Attention Deficit Hyperactivity Disorder (ADHD). However, it should be noted that ADHD is a non-discriminatory disorder affecting not only youth but people of every age, gender, IQ, religious and socio-economic background. In 2011, the Centers for Disease Control and Prevention reported that the percentage of children in the United States who have ever been diagnosed with ADHD is now 9.5%. Boys are diagnosed two to three times as often as girls.

One intervention that has been investigated for improving cognitive function in both healthy individuals and people with cognitive dysfunction is widely known as “brain training”. Brain training generally utilizes cognitive tasks, in many forms including games, to attempt to improve cognitive abilities. Certain approaches to brain training have particularly focused on utilizing computerized brain training software and Brain-computer interfaces (BCI), systems that mediate signaling between the brain and various technological devices. In 2009 the market for brain health software was estimated at $600 million and rapidly grew to $1 billion by the end of 2012. Researchers forecast the brain training software market to reach $4-10 billion by 2020.

Conclusions as to whether brain training is actually effective in having a meaningful positive impact on cognitive ability are mixed. Individual studies have shown statistically significant improvements in subject performance on standardized cognitive assessments following brain training programs. However, review of the evidence by The Stanford Center on Longevity and the Berlin Max Planck Institute for Human Development suggests that, while improvements may be seen in practiced skills, it remains unclear whether such improvements extend to other more broad cognitive areas and/or persist over time.

### SUMMARY

Aspects of the present disclosure include methods that include: presenting a cognitive task to a subject, wherein presenting the cognitive task may include presenting a stimulus or sequence of stimuli to the subject; monitoring neural activity of the subject during the presenting of the cognitive task, wherein the neural activity may include neural activity underlying one or more stimulus-related events, and the monitoring is time-locked to the one or more stimulus-related events; determining a neural performance level of the subject based on the neural activity underlying the one or more stimulus-related events; and adapting the cognitive task based on the neural performance level.

According to certain embodiments, the one or more stimulus-related events may include information processing, the information processing including cognitive processing and sensory processing.

According to certain embodiments, determining a neural performance level of the subject may be based on neural activity underlying the cognitive processing, the sensory processing, or both.

According to certain embodiments, adapting the cognitive task based on the neural performance level may include adapting an aspect of the cognitive task relating to cognitive processing, sensory processing, or both.

According to certain embodiments, presenting the cognitive task may include presenting a cue prior to presenting the stimulus or sequence of stimuli to the subject.

According to certain embodiments, the one or more stimulus-related events may include stimulus anticipation.

According to certain embodiments, determining a neural performance level of the subject may be based on neural activity underlying the stimulus anticipation.

According to certain embodiments, adapting the cognitive task based on the neural performance level may include adapting an aspect of the cognitive task relating to stimulus anticipation.

According to certain embodiments, the cognitive task requires the subject to respond to the stimulus.

According to certain embodiments, the one or more stimulus-related events may include response preparation.

According to certain embodiments, determining a neural performance level of the subject may be based on neural activity underlying the response preparation.

According to certain embodiments, adapting the cognitive task based on the neural performance level may include adapting an aspect of the cognitive task relating to response preparation.

According to certain embodiments, the cognitive task targets an aspect of cognition selected from the group consisting of: attention, working memory, task-switching, goal management, target search, target discrimination, and any combination thereof.

According to certain embodiments, the cognitive task is an attention task, a selective attention task, a selective attention task requiring the subject to discriminate target information from distractions, etc.

According to certain embodiments, the stimulus or sequence of stimuli may include a visual stimulus, an auditory stimulus, a tactile stimulus, an olfactory stimulus, or any combination thereof.

According to certain embodiments, the monitoring may include measuring neural activity of the subject by electroencephalography (EEG), functional magnetic resonance imaging (fMRI), near-infrared spectroscopy (NIRS), electrocortocography (ECoG), or a combination thereof, as the subject performs the cognitive task.

According to certain embodiments, the monitoring may include co-registering the neural activity of the subject with a 3-dimensional (3D) structural model of the subject's brain, including e.g., producing the 3D model of the subject's brain by performing a magnetic resonance imaging (MRI) structural brain scan on the subject prior to or during the presenting of the cognitive task.

According to certain embodiments, the method may include providing an indication to the subject of the subject's neural performance level, including e.g., an award.

According to certain embodiments, the subject has a cognitive deficit selected from the group consisting of: attention deficit hyperactivity disorder (ADHD), post-traumatic stress disorder (PTSD), major depressive disorder, dementia, or a combination thereof.

Aspects of the present disclosure include a system for neural activity detection and adaptive training, the system including: a user interface; a neural activity detector; a computing device including a non-transitory computer readable medium storing instructions that, when executed, cause the computing device to: present, through the user interface, a first cognitive task to a subject comprising a stimulus or sequence of stimuli to generate stimulus-related events in the brain of the subject; receive electrical signals from the neural activity detector during the presentation of the cognitive task that represents neural activity underlying the stimulus-related events in the brain of the subject; map the electrical signals in real-time onto a 3D model of the subject's brain to locate the neural activity; measure the strength of the located neural activity; determine a neural performance level of the subject based on the measured neural activity; present, through the user interface, a second cognitive task to the subject adapted according to the determined neural performance level.

According to certain embodiments, the user interface may include a display device adapted to relay a visual stimulus of the first and second cognitive tasks to the subject.

According to certain embodiments, the user interface may include an auditory device adapted to relay an audible stimulus of the first and second cognitive tasks to the subject.

According to certain embodiments, the user interface may include a tactile stimulator adapted to relay a tactile stimulus of the first and second cognitive tasks to the subject.

According to certain embodiments, the user interface may include an olfactory stimulator adapted to relay an olfactory stimulus of the first and second cognitive tasks to the subject.

According to certain embodiments, the user interface may include a taste stimulator adapted to relay a taste stimulus of the first and second cognitive tasks to the subject.

According to certain embodiments, the neural activity detector may include a device selected from the group consisting of: an electroencephalogram (EEG) device, a functional magnetic resonance imaging (fMRI) device, a near-infrared spectroscopy (NIRS) device, an electrocortocography (ECoG) device, and a combination thereof.

According to certain embodiments, the 3D model of the subject's brain may be generated from a magnetic resonance imaging (MRI) structural brain scan of the subject's brain.

According to certain embodiments, the system may further include a MRI scanner and the non-transitory computer readable medium further stores instructions that, when executed, cause the computing device to trigger the MRI scanner to generate the MRI structural brain scan of the subject's brain.

According to certain embodiments, the non-transitory computer readable medium further stores instructions that, when executed, cause the computing device to trigger the user interface to provide feedback to the subject based on the neural performance level of the subject.

According to certain embodiments, the user interface may further include a user input device adapted to allow the subject to input a behavioral response to the stimulus or sequence of stimuli.

According to certain embodiments, the non-transitory computer readable medium further stores instructions that, when executed, cause the computing device to assess the subject's behavioral performance level on the cognitive tasks and adapt the cognitive task based on both the neural performance level and the behavioral performance level.

According to certain embodiments, the non-transitory computer readable medium further stores instructions that, when executed, cause the computing device to trigger the user interface to provide feedback to the subject based on the behavioral performance level of the subject.

### BRIEF DESCRIPTION OF THE FIGURES

FIG. 1 depicts a schematic representation of an adaptive neural-performance based closed loop Cognitive Brain-Computer Interface (CBCI) according to one embodiment of the instant disclosure.

FIG. 2 depicts a schematic of the experimental flow of a trial of a closed loop neural CBCI cognitive training as described herein.

FIG. 3 depicts a schematic representation of a CBCI diagnostic task as described herein.

FIG. 4 depicts a schematic representation of a CBCI closed loop task as described herein.

FIG. 5 provides the behavioral cognitive efficiency and neural performance score data for subjects who underwent CBCI training sessions, showing improvement trends in both behavior and neural performance scores.

FIG. 6 depicts a schematic of a trial of CBCI training in children with ADHD.

FIG. 7 provides a schematic representation of a system according to one embodiment of the instant disclosure.

### DETAILED DESCRIPTION

Provided are methods of assessing and/or training cognitive fitness. Aspects of the instant methods generally relate to identifying and observing neural activity that underlies an event occurring in response to the stimulus or sequence of stimuli of a cognitive task performed by a subject. As such, the instant methods generally include presenting a cognitive task to a subject that includes a stimulus or sequence of stimuli, and monitoring the neural activity of the subject during performance of the cognitive task. Monitoring of such neural activity may be used, at least in part, to determine a neural performance level of a subject which may, in turn, be used in various ways including e.g., as an assessment of cognitive fitness, to tailor a subsequently presented cognitive task to train the cognitive fitness of the subject, etc. Systems and computer readable media for practicing the methods of the present disclosure are also provided.

Before the methods of the present disclosure are described in greater detail, it is to be understood that the methods are not limited to particular embodiments described, as such may, of course, vary. It is also to be understood that the terminology used herein is for the purpose of describing particular embodiments only, and is not intended to be limiting, since the scope of the methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within by the methods. The upper and lower limits of these smaller ranges may independently be included in the smaller ranges and are also encompassed within by the methods, subject to any specifically excluded limit in the stated range. Where the stated range includes one or both of the limits, ranges excluding either or both of those included limits are also included in the methods.

Unless defined otherwise, all technical and scientific terms used herein have the same meaning as commonly understood by one of ordinary skill in the art to which the methods belong. Although any methods similar or equivalent to those described herein can also be used in the practice or testing of the methods, representative illustrative methods, computer readable media and devices are now described.

Any publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, which are, for clarity, described in the context of separate embodiments, may also be provided in combination in a single embodiment. Conversely, various features of the methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or devices. In addition, all sub-combinations listed in the embodiments describing such variables are also specifically embraced by the present methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Definitions

When describing the methods and compositions of the present disclosure, the following terms include the following meanings unless otherwise indicated within the present disclosure, but the terms are not to be understood to be limited to their accompanying meaning as rather it is to be understood to encompass any meaning in accordance with the teachings and present disclosure.

The term “cognition”, as used herein, can include, but is not limited to, domains such as perception, attention, memory, motor function, problem solving, language processing, decision making and intelligence.

The term “task” refers to a behavior to be accomplished by an individual who provides a response to a particular stimulus that may include a goal and/or objective. For example, the individual may be instructed to perform a specific behavior to achieve a particular goal. The “task” can serve as the baseline cognitive function that is being performed and, optionally, measured, which induces a particular neural activity, including e.g., activity in a particular brain region. Thus, a “task” often refers to the main behavior that an individual is instructed to perform, which will include a mental component and may or may not include a physical component. For example, in some instances, a task may include a mental component of identifying or recognizing a particular stimulus and may or may not require a physical component of responding to the mental component, e.g., indicating that the stimulus has been identified or recognized, e.g., by performing a physical action such as pressing a button or verbally identifying the stimulus.

### Methods

The present disclosure provides methods of cognitive fitness assessment, methods of cognitive fitness training, and methods that combine cognitive fitness assessment and cognitive fitness training. The instant methods will generally include monitoring of neural activity of a subject performing a cognitive task such that, upon being presented with a stimulus or sequence of stimuli of the cognitive task, the associated stimulus-related events and their underlying neural activity may be identified and observed. Such monitoring of neural activity in a subject performing a cognitive task may be performed for a variety of reasons, as discussed in greater detail below, including but not limited to e.g., to determine the neural performance level of the subject.

Methods of the instant disclosure find use in assessing the neural performance of a subject and, in some instances, enhancing a subject's neural performance through cognitive training that includes successive neural performance assessments and adaptive training. Assessments of neural performance of the instant disclosure are based, at least in part, on observed neural activity occurring in a subject's brain while a subject performs a cognitive task. In some instances, the neural performance of a subject may be described in relative terms as a neural performance “level”. A neural performance level may be relative to various standards including but not limited to, the level of a subject's prior performance, the level equivalent to an average healthy subject, the level equivalent to an average unhealthy subject (e.g., a subject having a particular condition, e.g., a subject with cognitive impairment, a subject with an attention disorder, etc.). Determination of a subject's neural performance level may be utilized to assess the subject's cognitive ability for a variety of purposes including e.g., to detect cognitive impairment, to determine that the subject is cognitively normal, as part of a cognitive training program designed to enhance cognitive ability, as part of a cognitive training program designed to enhance neural performance, etc.

In some embodiments, the instant methods include presenting a subject with a cognitive task that includes presenting a stimulus to the subject and monitoring the neural activity of the subject such that the monitored neural activity is “time-locked” to the presentation of the stimulus. As used herein, the term “time-locked” refers to the association of two events in time including e.g., the association of a presented stimulus occurring at a particular time with the monitored neural activity occurring at that time, i.e., at the same time the stimulus is presented. Accordingly, by monitoring neural activity in a time-locked manner, the neural activity associated with the presentation of the stimulus may be determined.

The output of time-locked neural activity monitoring may be accessible in “real-time”. For example, real-time monitoring may include but is not limited to e.g., where the time-locked neural activity is accessible on a display that presents an instant or near instant readout (e.g., a delay of less than 1 sec, a delay of less than 900 ms, a delay of less than 800 ms, a delay of less than 700 ms, a delay of less than 600 ms, a delay of less than 500 ms, a delay of less than 400 ms, a delay of less than 300 ms, a delay of less than 200 ms, a delay of less than 100 ms, a delay of less than 50 ms, etc.) of the monitored neural activity while the cognitive task is being performed. Real-time monitoring is not, however, so limited and may e.g., also include where the readout of the monitoring is not displayed but is instead processed and fed in real-time back into the cognitive task, e.g., to adapt the cognitive task based on the monitored neural activity in real-time. In addition, real-time display monitoring and non-displayed real-time monitoring, e.g., as used for feedback into the method, are not mutually exclusive. For example, certain embodiments may include a combination of displayed real-time monitoring and non-displayed real-time monitoring.

The output of time-locked neural activity monitoring may be accessible “post hoc”. Time-locked neural activity monitoring accessible post hoc may include where the time at which a stimulus was presented and the neural activity monitored at the same time the stimulus is presented are provided at some period of time (e.g., greater than 1 second) after the stimulus presentation and simultaneous monitoring. A useful period of time when the monitoring is accessible after the stimulus is presented will vary and may range from one second to an hour or more, including but not limited to e.g., from 1 second to 5 seconds, 1 second to 10 seconds, 1 second to 30 seconds, 1 second to 1 min, 10 seconds to 30 seconds, 10 seconds to 1 min, 30 seconds to 1 min, 30 seconds to one hour or more, 1 min to 1 hour or more, 1 hour to 24 hours, 1 hour to 12 hours, etc. Monitoring accessible post hoc may include, in some instances, where the readout of the monitoring is displayed (e.g., “post hoc displayed monitoring”) and/or where the readout of the monitoring is not displayed (e.g., “post hoc non-displayed monitoring”). Accordingly, post hoc accessible monitoring may provide after the fact visual feedback of stimulus-related neural activity, non-visual feedback of stimulus-related neural activity and combinations thereof including where the post hoc monitoring readout is not displayed but is instead processed and fed in back into the cognitive task, e.g., to adapt the cognitive task based on the monitored neural activity.

Whether the readout of monitored neural activity is available in real-time or post hoc, time-locking of the monitored neural activity to a stimulus-related event allows for a clear association to be drawn between the event and the identified or observed neural activity. The term “stimulus-related event”, and often simply “event”, as used herein generally refers to any passive or active response, or lack thereof, of the subject to the presentation of a stimulus or a series of stimuli of a cognitive task. For example, a passive response may be the activation of a neuron, a neural pathway, a brain region, etc., resulting solely from the presentation/application of the stimulus to the subject. As such, in some instances, a stimulus-related event may include all or a portion of the neural activity occurring as a response to the presentation or application of the stimulus. Accordingly, in some instances, a stimulus-related event may be an involuntary response including e.g., an involuntary passive response.

In some instances, a stimulus-related event may include the absence of a predicted response of a subject. For example, a stimulus-related event may include the absence of a subject's predicted response to a visual, auditory, tactile, olfactory or taste stimulus meant to evoke a particular response (e.g., a target identification response, an emotional response, etc.). Accordingly, in some instances, a stimulus-related event may include a time period following a stimulus at which point the subject's neural activity, even in the absence of a response, is determined and time-locked to the stimulus.

In some instances, a stimulus-related event may refer to an active response of the subject resulting from the presentation of the stimulus to the subject or the application of the stimulus to the subject. Thus, a stimulus-related event may include a subject's actions in response to a presented or applied stimulus. For example, a subject may perform a physical action in response to a stimulus such as e.g., pressing a button, speaking a word, making a sound, moving a particular body part, walking, jumping, turning, sitting, stopping a particular action, etc. In some instances, a subject may perform a mental action in response to a stimulus such as e.g., thinking about a particular subject, performing a mental action (e.g., arithmetic, memory, verbal reasoning, etc.), preventing a mental action (e.g., ignoring distractors). When performing a mental action the subject may or may not be subsequently asked to perform a physical action to report the result of the mental action.

As used herein, a stimulus-related event may include an individual cognitive component of a task or may include a plurality of individual cognitive components of a task. For example, in some instances, a stimulus-related event may include an individual cognitive component of a complex task involving multiple responses to a stimulus. As an illustration, a subject may be presented with a stimulus requiring visual recognition of the stimulus, some cognitive processing of the stimulus (e.g., comparison to a reference), and a physical action to report the result of the cognitive processing (e.g., pressing a button to indicate whether the stimulus is the same or different as compared to the reference). Accordingly, one or more components of the complex task, e.g., the visual recognition, the cognitive processing, and/or the physical action may be considered stimulus-related events for which neural activity may be independently monitored.

As will be readily understood, the individual components of a cognitive task, which may be utilized as stimulus-related events, are not limited to those described in the above illustration and may include any passive or active component of a cognitive task, including but not limited to those active and passive components of the cognitive tasks described herein. Accordingly, individual components of a cognitive task may be defined as a stimulus-related event such that monitored neural activity may be associated (e.g., in a time-locked manner) with an appropriate stimulus-related event for making an assessment of cognitive fitness and/or training cognitive fitness as desired.

In some instances, a stimulus-related event may include a plurality, or grouping, of individual cognitive components of a task. For example, two or more components of a cognitive task may be combined into one stimulus-related event. Using the above illustration as an example, a stimulus-related event may be considered to include e.g., both the visual recognition of the stimulus and the cognitive processing of the stimulus, both the cognitive processing of the stimulus and the physical action to report the result of the cognitive processing, or all of the components of the task (e.g., the recognizing, the processing and the reporting). Accordingly, components of a cognitive task may be grouped as appropriate into a defined stimulus-related event such that monitored neural activity may be associated (e.g., in a time-locked manner) with an appropriate stimulus-related event for making an assessment of cognitive fitness and/or training cognitive fitness as desired.

Useful stimulus related-events, either alone or in combination with other stimulus-related events include but are not limited to e.g., stimulus induced information processing events (e.g., cognitive processing, sensory processing, etc.), stimulus anticipation, stimulus induced response preparation, distractor induced interference, stimulus induced memory recall, stimulus induced memory formation, stimulus induced task-switching, stimulus or distractor induced goal management, stimulus induced target search, stimulus induced target discrimination, combinations thereof, and the like.

In some embodiments, the neural activity of a subject may be detected before and/or when a subject is presented with a stimulus and the subject's neural activity may continue to be monitored for some time after the stimulus has been presented. Whether the monitoring is begun prior to presenting the stimulus and the length of the monitoring will vary depending on a number of factors including but not limited to the particular stimulus and when the subject would be expected to mount a response to the particular stimulus. For example, in some instances, e.g., where a subject is expected to mount an immediate response or a response in a short time following the stimulus, monitoring may be begun prior to the presentation of the stimulus. In some instances, e.g., where a subject is expected not to mount an immediate response, monitoring may be begun at the time the stimulus is presented or following the presentation of the stimulus but before the response is expected. In some instances, monitoring may be continuous, e.g., occurring during the entire cognitive task and may or may not include a period before and/or after the cognitive task has been performed.

Accordingly, the length of the monitoring period will vary depending on the particular cognitive task to be performed and the nature of the assessment and/or training. As such useful monitoring periods will range from milliseconds or less to days or more including but not limited to e.g., one millisecond to one week, one second to one week, one hour to one week, one day to one week, one millisecond to one second, one millisecond to one minute, one millisecond to one hour, one second to one day, one second to 12 hours, one second to one hour, one minute to one day, one minute to 12 hours, one minute to one hour, etc.

As mentioned above, in some instances, monitoring may be performed prior to presentation of a stimulus. Such pre-stimulus monitoring may be performed for a variety of reasons. For example, in some instances, pre-stimulus monitoring may provide a “baseline” of neural activity, e.g., to which a stimulus-related “spike” or increase or “dip” or decrease in neural activity may be compared. In some instances, pre-stimulus monitoring may allow for the detection of neural activity associated with a pre-stimulus activity including but not limited to e.g., anticipation behavior, false response (e.g., “false start”) behavior, etc. Pre-stimulus monitoring may or may not include a “cue” presented to the subject, e.g., to indicate that a stimulus is soon to be presented. Accordingly, pre-stimulus monitoring may be initiated before, during or after the presentation of a cue and/or in the absence of a cue.

In some instances, neural activity monitoring may be continued for a predetermined period of time including e.g., where the predetermined period of time is relative to some aspect of the cognitive task including but not limited to e.g., the time necessary for an average person to perform the cognitive task. In some instances, the length of monitoring may be relative to some event of the task including e.g., the presentation of the stimulus, the presentation of a cue, the subject's response to the stimulus, etc. In some instances, the predetermined period of time is not related to another aspect of the cognitive task and may be referred to as a “set period of time” including but not limited to e.g., one second, one minute, five minutes, 10 minutes, 15 minutes, 30 minutes, 45 minutes, one hour, two hours, three hours, four hours, 6 hours, 8 hours, 10 hours, 12 hours, one day, two days, three days, four days, a week, a month, etc. In some instances, the monitoring may be continued until some “goal” is achieved including but not limited to e.g., attainment of a desired cognitive fitness level, attainment of a desired neural performance level, attainment of a desired behavioral response, etc. In some instances, the monitoring may be continued until some adverse event is encountered including but not limited to e.g., fatigue, disinterest, cessation of improvement, reversal of improvement, etc.

According to the methods as described herein, in some embodiments, neural activity monitoring during cognitive testing may be employed as the sole method for determining performance of a cognitive task. In some embodiments, neural activity monitoring may be combined with one or more additional methods for determining performance of a cognitive task. Additional methods for determining performance of a cognitive task include but are not limited to e.g., cognitive assessment (e.g., as determined based on the performance of the cognitive task itself (including e.g., the accuracy with which the task is performed, the precision with which the task is performed, the speed with which the task is performed, some combination thereof, etc.)), performance of a standardized cognitive assessment, neurophysiological parameters (including e.g., autonomic function parameters (e.g., heart rate variability (HRV), inspiration to expiration ratio (I:E ratio), 30:15 ratio, postural challenge test, sustained handgrip test, etc.), pain perception parameters, etc.), and the like.

Such additional measures may, in some instances, be performed in parallel with neural activity monitoring and/or before or after monitoring (e.g., to provide a baseline and/or provide a follow-up assessment). In some instances, an assessment of a subject's performance on a cognitive task may include both a neural activity component and one or more components that include an additional parameter. For example, in some instances, a subject's performance on a cognitive task may include a neural activity component and one or more cognitive performance parameters including but not limited to e.g., the accuracy with which the cognitive task was performed, the precision with which the cognitive task was performed, the speed with which the cognitive task was performed, and combinations thereof. In some instances, a subject's performance on a cognitive task may include a neural activity component and one or more neurophysiological parameters. In some instances, a subject's performance on a cognitive task may include a neural activity component and one or more cognitive performance parameters and one or more neurophysiological parameters.

According to the methods described herein, in some embodiments, a subject's neural performance may be based solely on monitored neural activity. In some embodiments, a subject's neural performance may be based on some combination of monitored neural activity and one or more additional parameters including e.g., those described above.

### Detecting Neural Activity

According to embodiments described herein, monitoring of neural activity includes the detection of neural activity for some period of time, as described in more detail above. By “detecting neural activity”, as used herein, is meant sensing a change in the activity state of one or more brain regions of a subject wherein such sensing may be performed by a variety of means including but not limited to electromagnetic sensing, metabolic sensing, vascular/blood flow sensing, etc.

Whereas neural activity may be detected directly, e.g., by directly measuring the electrical potential or current generated by a neuron or a collection of neurons, neural activity may also be detected indirectly through the use of various methods such as, but not limited to, those that indirectly detect changes in neural activity within the brain, at the surface of the brain, at the surface of the scalp, and/or beyond the surface of the scalp. Indirect neural activity detection methods include but are not limited to e.g., those that detect electromagnetic waves at the surface of the brain, those that detect electromagnetic waves at the surface of the scalp, those that detect the movement of detectable agents within the brain and/or associated vasculature using an imaging device, those that detect the uptake of detectable metabolites by the brain, and the like.

Given the diversity of neural activity detection techniques, resolution may vary. For example, depending on the particular technique employed neural activity may be detected at whole brain resolution, at brain lobe resolution, at brain structure resolution, a neural pathway resolution, at nerve fiber resolution, etc. Different neural activity detection techniques may be utilized individually or in combination. Accordingly, a particular technique may be employed in the subject methods provided the resolution of the technique is sufficient for detection of neuronal activity at a level of resolution corresponding to the desired assessment to be made. For example, where the assessment is made at the level of whole brain activity a low resolution technique may be employed, however, where the assessment is made at the level of neural pathway activity a high resolution technique may be employed.

In some instances, neural activity may be detected at the level of the whole brain. For example, whole brain activity, e.g., as detected by a whole brain EEG recording, may be determined and be compared to a prior whole brain reading or a reference whole brain reading to assess whether the whole brain neural activity is increased or decreased relative to the prior reading or reference activity.

In some instances, neural activity may be detected at the level of brain lobes. For example, one or more neural activity detection devices may be employed to measure the neural activity of a particular brain lobe or sub-portion thereof and the measurement may be compared to a prior measurement or a reference measurement to determine if the measured activity is increased, decreased, normal, abnormal, etc. Brain lobes that could be measured include but are not limited to the frontal lobe (either the entire frontal lobe or portions thereof including but not limited to e.g., Superior Frontal, Rostral Middle Frontal, Caudal Middle Frontal, Pars Opercularis, Pars Triangularis, and Pars Orbitalis, Lateral Orbitofrontal, Medial Orbitofrontal, Precentral, Paracentral, Frontal Pole, combinations thereof, and the like), parietal lobe (either the entire parietal lobe or portions thereof including but not limited to e.g., Superior Parietal, Inferior Parietal, Supramarginal, Postcentral, Precuneus, combinations thereof, and the like), temporal lobe (either the entire temporal lobe or portions thereof including but not limited to e.g., Superior Temporal, Middle Temporal, Inferior Temporal, Banks of the Superior Temporal Sulcus, Fusiform, Transverse Temporal, Entorhinal, Temporal Pole, Parahippocampal, combinations thereof, and the like) and occipital lobe (either the entire occipital lobe or portions thereof including but not limited to e.g., Lateral Occipital, Lingual, Cuneus, Pericalcarine, combinations thereof, and the like).

In some instances, neural activity may be detected at the level of the brain structures. For example, one or more neural activity detection devices may be employed to measure the neural activity of a particular brain structure or sub-portion thereof and the measurement may be compared to a prior measurement or a reference measurement to determine if the measured activity is increased, decreased, normal, abnormal, etc. Brain structures that could be measured include but are not limited to Hindbrain structures (e.g., Myelencephalon structures (e.g., Medulla oblongata, Medullary pyramids, Olivary body, Inferior olivary nucleus, Respiratory center, Cuneate nucleus, Gracile nucleus, Intercalated nucleus, Medullary cranial nerve nuclei, Inferior salivatory nucleus, Nucleus ambiguous, Dorsal nucleus of vagus nerve, Hypoglossal nucleus, Solitary nucleus, etc.), Metencephalon structures (e.g., Pons, Pontine cranial nerve nuclei, chief or pontine nucleus of the trigeminal nerve sensory nucleus (V), Motor nucleus for the trigeminal nerve (V), Abducens nucleus (VI), Facial nerve nucleus (VII), vestibulocochlear nuclei (vestibular nuclei and cochlear nuclei) (VIII), Superior salivatory nucleus, Pontine tegmentum, Respiratory centres, Pneumotaxic centre, Apneustic centre, Pontine micturition center (Barrington's nucleus), Locus coeruleus, Pedunculopontine nucleus, Laterodorsal tegmental nucleus, Tegmental pontine reticular nucleus, Superior olivary complex, Paramedian pontine reticular formation, Cerebellar peduncles, Superior cerebellar peduncle, Middle cerebellar peduncle, Inferior cerebellar peduncle, Fourth ventricle, Cerebellum, Cerebellar vermis, Cerebellar hemispheres, Anterior lobe, Posterior lobe, Flocculonodular lobe, Cerebellar nuclei, Fastigial nucleus, Interposed nucleus, Globose nucleus, Emboliform nucleus, Dentate nucleus, etc.)), Midbrain structures (e.g., Tectum, Corpora quadrigemina, inferior colliculi, superior colliculi, Pretectum, Tegmentum, Periaqueductal gray, Parabrachial area, Medial parabrachial nucleus, Lateral parabrachial nucleus, Subparabrachial nucleus (Kölliker-Fuse nucleus), Rostral interstitial nucleus of medial longitudinal fasciculus, Midbrain reticular formation, Dorsal raphe nucleus, Red nucleus, Ventral tegmental area, Substantia nigra, Pars compacta, Pars reticulata, Interpeduncular nucleus, Cerebral peduncle, Crus cerebri, Mesencephalic cranial nerve nuclei, Oculomotor nucleus (III), Trochlear nucleus (IV), Mesencephalic duct (cerebral aqueduct, aqueduct of Sylvius), etc.), Forebrain structures (e.g., Diencephalon, Epithalamus structures (e.g., Pineal body, Habenular nuclei, Stria medullares, Taenia thalami, etc.) Third ventricle, Thalamus structures (e.g., Anterior nuclear group, Anteroventral nucleus (aka ventral anterior nucleus), Anterodorsal nucleus, Anteromedial nucleus, Medial nuclear group, Medial dorsal nucleus, Midline nuclear group, Paratenial nucleus, Reuniens nucleus, Rhomboidal nucleus, Intralaminar nuclear group, Centromedial nucleus, Parafascicular nucleus, Paracentral nucleus, Central lateral nucleus, Central medial nucleus, Lateral nuclear group, Lateral dorsal nucleus, Lateral posterior nucleus, Pulvinar, Ventral nuclear group, Ventral anterior nucleus, Ventral lateral nucleus, Ventral posterior nucleus, Ventral posterior lateral nucleus, Ventral posterior medial nucleus, Metathalamus, Medial geniculate body, Lateral geniculate body, Thalamic reticular nucleus, etc.), Hypothalamus structures (e.g., Anterior, Medial area, Parts of preoptic area, Medial preoptic nucleus, Suprachiasmatic nucleus, Paraventricular nucleus, Supraoptic nucleus (mainly), Anterior hypothalamic nucleus, Lateral area, Parts of preoptic area, Lateral preoptic nucleus, Anterior part of Lateral nucleus, Part of supraoptic nucleus, Other nuclei of preoptic area, median preoptic nucleus, periventricular preoptic nucleus, Tuberal, Medial area, Dorsomedial hypothalamic nucleus, Ventromedial nucleus, Arcuate nucleus, Lateral area, Tuberal part of Lateral nucleus, Lateral tuberal nuclei, Posterior, Medial area, Mammillary nuclei (part of mammillary bodies), Posterior nucleus, Lateral area, Posterior part of Lateral nucleus, Optic chiasm, Subfornical organ, Periventricular nucleus, Pituitary stalk, Tuber cinereum, Tuberal nucleus, Tuberomammillary nucleus, Tuberal region, Mammillary bodies, Mammillary nucleus, etc.), Subthalamus structures (e.g., Thalamic nucleus, Zona incerta, etc.), Pituitary gland structures (e.g., neurohypophysis, Pars intermedia (Intermediate Lobe), adenohypophysis, etc.), Telencephalon structures, white matter structures (e.g., Corona radiata, Internal capsule, External capsule, Extreme capsule, Arcuate fasciculus, Uncinate fasciculus, Perforant Path, etc.), Subcortical structures (e.g., Hippocampus (Medial Temporal Lobe), Dentate gyrus, Cornu ammonis (CA fields), Cornu ammonis area 1, Cornu ammonis area 2, Cornu ammonis area 3, Cornu ammonis area 4, Amygdala (limbic system) (limbic lobe), Central nucleus (autonomic nervous system), Medial nucleus (accessory olfactory system), Cortical and basomedial nuclei (main olfactory system), Lateral[disambiguation needed] and basolateral nuclei (frontotemporal cortical system), Claustrum, Basal ganglia, Striatum, Dorsal striatum (aka neostriatum), Putamen, Caudate nucleus, Ventral striatum, Nucleus accumbens, Olfactory tubercle, Globus pallidus (forms nucleus lentiformis with putamen), Subthalamic nucleus, Basal forebrain, Anterior perforated substance, Substantia innominata, Nucleus basalis, Diagonal band of Broca, Medial septal nuclei, etc.), Rhinencephalon structures (e.g., Olfactory bulb, Piriform cortex, Anterior olfactory nucleus, Olfactory tract, Anterior commissure, Uncus, etc.), Cerebral cortex structures (e.g., Frontal lobe, Cortex, Primary motor cortex (Precentral gyrus, M1), Supplementary motor cortex, Premotor cortex, Prefrontal cortex, Gyri, Superior frontal gyrus, Middle frontal gyrus, Inferior frontal gyrus, Brodmann areas: 4, 6, 8, 9, 10, 11, 12, 24, 25, 32, 33, 44, 45, 46, 47, Parietal lobe, Cortex, Primary somatosensory cortex (S1), Secondary somatosensory cortex (S2), Posterior parietal cortex, Gyri, Postcentral gyrus (Primary somesthetic area), Other, Precuneus, Brodmann areas 1, 2, 3 (Primary somesthetic area); 5, 7, 23, 26, 29, 31, 39, 40, Occipital lobe, Cortex, Primary visual cortex (V1), V2, V3, V4, V5/MT, Gyri, Lateral occipital gyrus, Cuneus, Brodmann areas 17 (V1, primary visual cortex); 18, 19, Temporal lobe, Cortex, Primary auditory cortex (A1), secondary auditory cortex (A2), Inferior temporal cortex, Posterior inferior temporal cortex, Superior temporal gyrus, Middle temporal gyrus, Inferior temporal gyrus, Entorhinal Cortex, Perirhinal Cortex, Parahippocampal gyrus, Fusiform gyrus, Brodmann areas: 9, 20, 21, 22, 27, 34, 35, 36, 37, 38, 41, 42, Medial superior temporal area (MST), Insular cortex, Cingulate cortex, Anterior cingulate, Posterior cingulate, Retrosplenial cortex, Indusium griseum, Subgenual area 25, Brodmann areas 23, 24; 26, 29, 30 (retrosplenial areas); 31, 32, etc.)).

In some instances, neural activity may be detected at the level of the neural pathways. For example, one or more neural activity detection devices may be employed to measure the neural activity of a particular neural pathway or sub-portion thereof and the measurement may be compared to a prior measurement or a reference measurement to determine if the measured activity is increased, decreased, normal, abnormal, etc. Neural pathways structures that could be measured include but are not limited to any neural pathways of those brain lobes and structures described above, Superior Longitudinal Fasciculus, Arcuate fasciculus, Cerebral peduncle, Corpus callosum, Pyramidal or corticospinal tract, Major dopamine pathways dopamine system, Mesocortical pathway, Mesolimbic pathway, Nigrostriatal pathway, Tuberoinfundibular pathway, Serotonin Pathways serotonin system, Raphe Nuclei, Norepinephrine Pathways, Locus coeruleus, etc.

In some instances, neural activity may be detected at the level of nerve fibers. For example, one or more neural activity detection devices may be employed to measure the neural activity of a particular nerve fiber and the measurement may be compared to a prior measurement or a reference measurement to determine if the measured activity is increased, decreased, normal, abnormal, etc.

In some instances, neural activity may be detected and/or determined according to a structure of a neuroanatomical atlas. Useful neuroanatomical atlases include print and electronic neuroanatomical atlases including but not limited to e.g., the Destrieux Atlas, the Desikan-Killiany Atlas, the DKT Atlas, and the like. In some instances, a region of neural activity, including e.g., a region analyzed in a method of the instant disclosure may be identified according to neuroanatomical labeling based on cortical parcellation e.g., as performed using one or more of the “FreeSurfer” utilities including but not limited to e.g., those based on various neuroanatomical atlases including but not limited to e.g., the Destrieux Atlas, the Desikan-Killiany Atlas, the DKT Atlas, and the like.

According to some embodiments, suitable approaches for detecting neural activity include, but are not limited to, electroencephalography (EEG), near-infrared spectroscopy (NIRS), optical imaging, functional magnetic resonance imaging (fMRI), electrocorticography (ECoG), and any combination thereof. Accordingly, useful devices for detecting neural activity include EEG devices, NIRS devices, optical imaging devices, fMRI devices, ECoG devices, and the like.

In some instances, neural activity of the subject is recorded using a EEG including but not limited to e.g., EEG device (e.g., an EEG cap, such as a 64-channel EEG cap (or “headset”)) worn by the subject to enable recording of voltage fluctuations resulting from ionic current flows within the neurons of the brain at desired intervals, including in real-time, during presentation of the cognitive task and the subject's response thereto.

In some instances, the method employed for detecting neural activity may be an invasive or minimally invasive method where a neural activity detection device, or a portion thereof, is implanted into the subject, including e.g., into the brain of the subject, onto the surface of the brain of the subject, under the skin of the scalp of the subject, etc. For example, in some instances, the electrodes of an ECoG device may be place or implanted onto the surface of the brain of the subject.

In some instances, the method employed for detecting neural activity may be a noninvasive method, i.e., where no device or portion thereof is implanted into the subject or under the skin of subject. For example, in some instances, neural activity may be monitored using a EEG device that contacts, but does not penetrate, the scalp of the subject or a noninvasive imaging device such as e.g., a fMRI.

In some instances, detected neural activity may be “co-registered” (i.e., “mapped”) onto a reference map or model of a brain. The reference map of the brain may be a general reference map or a subject-specific reference map. For example, in instances where a subject-specific reference map is used, the method may include mapping the subject's brain with one or more brain imaging techniques and overlaying the detected neural activity onto the subject-specific map. A reference brain map of a subject may be obtained prior to monitoring associated with a cognitive assessment and/or training. Alternatively, a reference brain map may be obtained during the monitoring of the herein described methods, including e.g., where the method of detecting neural activity simultaneously produces a subject-specific brain map (e.g., as in fMRI) or where a neural activity detection technique is combined with a second technique for brain imaging (e.g., combined MRI and EEG recording).

In some instances, neural activity detected using a high-density EEG is mapped in real-time or near real-time (e.g., computational lag of less than 1 second, including but not limited to e.g., less than 500 milliseconds, less than 400 milliseconds, less than 300 milliseconds, less than 200 milliseconds, between 200 to 100 milliseconds, etc.) onto a previously acquired Diffusion Tensor Imaging (DTI) 3D reconstruction of the subject's brain. By “high-density EEG” is meant at least 64-channel EEG, however, EEG sensor density may vary and may include, in some instances, greater than 64-channel EEG (e.g., 128-channel EEG), less than 64-channel EEG (e.g., 32-channel EEG, 24-channel EEG, etc.). In some instances, different physiologically relevant frequency bands may be differentiated including e.g., where only certain bands are displayed, where different bands are displayed at different intensities (including absolute intensities, relative intensities, threshold intensities, etc.), where different bands are displayed in different colors. Different physiologically relevant frequency bands include physiologically relevant alpha frequency bands (e.g., 8-12 Hz), physiologically relevant beta frequency bands (e.g., 12-20 Hz) and physiologically relevant theta frequency bands (e.g., 4-8 Hz). In some instances, before display, neural activity may be corrected for irrelevant interference including but not limited to e.g., ocular artifacts, muscular artifacts, etc. In some instances, effective connectivity may be and mapped and/or visualized calculated in real-time or near real-time onto a previously acquired reference brain model, including e.g., a DTI 3D reconstruction.

### Stimuli

Aspects of the instant methods include presenting a subject with a cognitive task that includes presenting the subject with a stimulus. As described above, the presentation of the stimulus may represent a stimulus-related event and/or may evoke or trigger a stimulus-related event such as a passive or active response by the subject. The monitored neural activity may be time-locked to the stimulus-related event allowing the assessment of neural activity specifically attributed to the stimulus-related event. Useful stimuli, as discussed in more detail below, will vary depending on the particular cognitive task employed and the desired assessment criteria.

For simplicity, a number of embodiments are herein described with the presentation of a single stimulus. However, as will be readily understood and is further described below, in many instances neural activity may be monitored during the presentation of a plurality of stimuli including but not limited to e.g., 2 or more stimuli, 3 or more stimuli, 4 or more stimuli, 5 or more stimuli, 6 or more stimuli, 7 or more stimuli, 8 or more stimuli, 9 or more stimuli, 10 or more stimuli, 11 or more stimuli, 12 or more stimuli, 13 or more stimuli, 14 or more stimuli, 15 or more stimuli, 16 or more stimuli, 17 or more stimuli, 18 or more stimuli, 19 or more stimuli, 20 or more stimuli, etc.

Multiple stimuli may be presented individually, e.g., with an intermission between them, or may be presented as a defined series of stimuli, e.g., without an intermission between two or more of the stimuli. In some instances, series of stimuli may be presented in “blocks”, e.g., without an intermission between the stimuli of the series but with an intermission between blocks. When presented as a series of stimuli, neural activity may be monitored before, during and/or after the series. As such, the monitoring may be time-locked to all or any portion of the series including but not limited to e.g., where the monitoring is time-locked to each stimulus of the series, to the first stimulus of the series, to the last stimulus of the series, to some interval of a portion of stimuli of the series (e.g., every other stimulus, every third stimulus, etc.), to a combination thereof, etc. It will be understood that, where appropriate, a stimulus described herein in the singular may be exchanged for a stimulus presented as a series of stimuli and vice versa.

A stimulus presented to an individual can be visual. A visual stimulus is made up of electromagnetic waves in the visible light spectrum and may be characterized by, for example: brightness, color, shape, surface texture, orientations (e.g. grating), location in a visual field, orthographic (e.g. textual), quantity, or motions, as well as properties of these characteristics. Visual stimuli may, in some instances, also be referred herein as graphical elements.

Each graphical element may be presented with a specific duration to an individual, e.g. for a fraction of a second (e.g., a millisecond, tens of milliseconds, hundreds of milliseconds, etc.), for a second or for a length between about 1 and about 2 seconds or for up to about 2 seconds or more. For example, a visual stimulus may be a geometric shape of a circle, or a red cone, the number “5”, or the like. Another example of a visual stimulus can be a specific human face, face of a specific age range, face of different ethnicities, or any parametric variations thereof.

A visual stimulus can also be an image that is rich so as to contain multiple shapes, colors, textures, etc., as seen in a photograph, digitally-generated picture, or moving video such as in movies or video games. A series of consecutive images can be presented so the individual would perceive the visual stimuli in a form of a motion picture.

Where visual stimuli are employed in a task, the task can be target discrimination for example. The task may involve instructing an individual to respond to a green circle whenever a green circle appears on the screen. Where other shapes or other colored circles are presented (e.g. green pentagon), the individual is not to respond. A target visual stimulus can also be a face of a child while the non-target stimulus is a face of an adult. As another example, a target visual stimulus can also be a visual image of a first animal (e.g., non-human animal) of a certain species while the non-target visual stimulus can be an animal of a species different from the first animal. A target visual stimulus may differ from a non-target visual stimulus in one or more of any properties inferred from herein.

The visual stimuli can also be presented in another type of discrimination task. The individual is presented a series of visual graphical elements and is instructed to respond to a target graphical element that does not belong in the same category as other non-target graphical elements in the series. In another similar task, the individual can be instructed to identify a part that does not appear in a correct location or orientation as other parts of an object that are presented to the individual in a series.

Where the task involves images presented as a motion picture, the task can involve visuomotor control. The individual would be instructed to control a moving object in an environment in which the colors, shapes, or any properties discussed above are changing. One example includes navigating a moving vehicle on a winding path or on a path with obstructions. Other examples can include clicking on specific objects that appear or move.

A stimulus presented to an individual can be auditory. An auditory stimulus refers to a sound and may be characterized by, for example: frequency, loudness (i.e. intensity), timbre, or any parametric combination of these or any other sound features. The duration of time an auditory stimulus is presented to an individual can be varied. For example, an auditory stimulus may be presented to an individual, e.g. for a fraction of a second (such as about 40 milliseconds (ms), about 50 ms, about 60 ms, about 70 ms or more), for a second or for a length between about 1 and about 2 seconds or for up to about 2 seconds or more. An example of a duration of an auditory stimulus presentation is about 100 ms.

A stimulus can also be spectrally-complex stimuli like vowels, phonemes, syllables, words, questions, or statements. A stimulus can also be presented by a voice and as such characterized by the presenting voice (e.g. call of a specific bird). The auditory stimulus can also be characterized by a waveform that is defined by amplitude (i.e. intensity or loudness), frequency, or any other sinusoidal properties.

Similarly to the visual stimulus discussed above in which a series of visual stimulus is perceived as a motion picture, a series of auditory stimulus can be perceived by the individual as a statement, a song, a narration, etc.

Where the task involves target discrimination, a target auditory stimulus can differ from a non-target auditory stimulus in any one or more of the characteristics, such as frequency, loudness, or timbre, as well as properties of these characteristics. For example, if they differ in frequency, the difference in frequency may be measured in hertz or octave. Hertz (Hz) measures the numbers of cycles per second in the sound wave while octave represents frequency as pitch. The frequency difference between a non-target auditory stimulus and a target auditory stimulus may be between about 0.01 to about 0.05%, between about 0.05% to about 0.1%, between about 0.1% to about 0.3%, between about 0.3% to about 0.5%, between about 0.5% to 1%, between about 1% to about 3%, between about 3% to about 6%, up to about 9% or more.

Where the difference between a non-target auditory stimulus and a target auditory stimulus differs in loudness, the difference can be expressed in sound pressure level (SPL) measured in decibels (dB) above a standard reference level. The standard reference level is about 20 μPa. For example, in a target discrimination task, an individual can be instructed to respond to only to a female voice.

In another task that involves auditory stimulus, the individual can be instructed to answer a question that is asked vocally or to repeat certain words or sounds in response to a spoken auditory stimulus.

Any of the characteristics of sound described above, and combinations thereof, can be one or more of the ways in which the target auditory stimulus may be used in the present methods.

A stimulus presented to an individual can be a tactile stimulus. Tactile stimuli are stimuli a subject can feel through the sense of touch. A tactile stimulus can be characterized by pressure, texture, temperature, hardness, softness, etc., or any combination of tactile characteristics. The duration of time a tactile stimulus is presented to an individual can be varied. For example, a tactile stimulus may be presented to an individual, e.g. for a fraction of a second (such as about 40 milliseconds (ms), about 50 ms, about 60 ms, about 70 ms or more), for a second or for a length between about 1 and about 2 seconds or for up to about 2 seconds or more. An example of a duration of a tactile stimulus presentation is about 100 ms.

Where the task involves target discrimination, a target tactile stimulus can differ from a non-target tactile stimulus in any one or more tactile characteristics, such as pressure, texture, temperature, hardness, softness, etc. For example, a subject may be presented with two or more tactile stimuli, either in parallel or series, and asked to identify the harder of the two stimuli, the hotter of the two stimuli, which stimulus is presented (e.g., touched to the subject) with greater pressure, etc.

A stimulus presented to an individual can be an olfactory stimulus. Olfactory stimuli are stimuli a subject can smell through the olfactory system of the nose. An olfactory stimulus can be characterized by strength or similarity to one or more aromas including but not limited to e.g., fragrant, fruity, citrus, woody/resinous, chemical, sweet, minty/peppermint, toasted/nutty, pungent, decayed, etc., or any combination of olfactory characteristics. The duration of time an olfactory stimulus is presented to an individual can be varied. For example, an olfactory stimulus may be presented to an individual, e.g. for a fraction of a second (such as about 40 milliseconds (ms), about 50 ms, about 60 ms, about 70 ms or more), for a second or for a length between about 1 and about 2 seconds or for up to about 2 seconds or more. An example of a duration of a olfactory stimulus presentation is about 100 ms.

Where the task involves target discrimination, a target olfactory stimulus can differ from a non-target olfactory stimulus in any one or more olfactory characteristics, such as strength or aroma. For example, a subject may be presented with two or more smell stimuli, either in parallel or series, and asked to identify the stronger of the two stimuli, the sweeter of the two stimuli, the more pungent of the two stimuli, etc.

A stimulus presented to an individual can be a taste stimulus. Taste stimuli are stimuli a subject can discriminate through the taste buds of the tongue. A taste stimulus can be characterized by strength or the relative contribution of one or more taste sensations including but not limited to e.g., sweet, bitter, sour, salty and umami, or any combination of taste characteristics. The duration of time a taste stimulus is presented to an individual can be varied. For example, a taste stimulus may be presented to an individual, e.g. for a fraction of a second (such as about 40 milliseconds (ms), about 50 ms, about 60 ms, about 70 ms or more), for a second or for a length between about 1 and about 2 seconds or for up to about 2 seconds or more. An example of a duration of a taste stimulus presentation is about 100 ms.

Where the task involves target discrimination, a target taste stimulus can differ from a non-target taste stimulus in any one or more taste characteristics, such as strength, sweetness, bitterness, sourness, saltiness or umami. For example, a subject may be presented with two or more taste stimuli, either in parallel or series, and asked to identify the stronger of the two stimuli, the sweeter of the two stimuli, the more bitter of the two stimuli, etc.

A combination of stimuli of different sensory systems may be presented to the individual in the methods of the instant disclosure. For example, both auditory and visual stimuli may be presented concurrently or in sequence. The series can also present a sequence of auditory and visual stimuli that can be synchronized or unsynchronized. In a discrimination task, a target set can contain either a target auditory or a target visual stimulus or both. For example, a target stimulus may be a combination of the visual stimulus of a green circle as well as the spoken word “circle” as the auditory stimulus. Any of the above stimuli may, in certain instances, also serve as a distractor in a cognitive task, e.g., where the stimulus is a non-target stimulus meant to interfere with completion of the goal of the cognitive task as in, e.g., interfere with a subject's sustained attention to or recognition of a target stimuli.

### Feedback

According to some embodiments, the methods include providing feedback. Feedback may include neural activity feedback including where the neural activity of a subject, including e.g., particular neural pathway activity, is detected and indicated to the subject. Neural activity feedback may be presented in any desired manner to the subject. For example, in some instances, neural feedback may be presented to an individual undergoing or having undergone a cognitive task as a visual representation of the neural activity, e.g., using lights and/or colors to identify the spatial and temporal positions of the neural activity on a two-dimensional or 3D model of the subject's brain. In some instances, neural feedback may be presented to a subject through an indication that a desired or an undesired neural activity has occurred, e.g., through the presentation of a positive sign (e.g., a “+” sign, a green light, a star, a “ding” or bell sound, etc.) when a desired neural activity has occurred and a negative sign (e.g., a “−” sign, a red light, a frowning face, a “buzzer” sound, etc.) when an undesired neural activity has occurred. Accordingly, neural feedback indicated to a subject may be direct, in that it identifies the activity on a representation of the brain, or indirect, in that it uses some sign a proxy for desired and/or undesired neural activity. In some instances, direct and indirect neural feedback may be combined, e.g., where a subject it provided with a visualization of their neural activity in a representation of their brain and signs indicating whether the neural activity is desired or undesired.

In some instances, neural feedback is presented to the subject, e.g., through a display or an audible indication (e.g., through speakers or a bell). However, neural activity feedback need not always be indicated to a subject. In some instances, the feedback of neural activity may be stored and/or directed back into a cognitive task without the subject being aware of the feedback. Whether or not the feedback is presented to the subject and/or whether the subject is aware of the feedback, feedback may be directed back into the cognitive task, e.g., to influence the type of stimulus presented next, the difficulty of the next task, etc.

Feedback in the methods described herein is not limited to neural activity feedback and may, in some instances, include behavioral feedback, i.e., positive and/or negative feedback generated in response to a subject's behavior and/or performance on the cognitive task and/or a portion thereof. Behavioral feedback may include e.g., positive feedback in response to a subject completing a task correctly (e.g., correctly identifying a target stimulus) and negative feedback in response to a subject completing a task incorrectly (e.g., incorrectly identifying a target stimulus).

Feedback (e.g., positive (e.g., rewards) and/or negative feedback) may be provided to the subject based on the subject's performance, including e.g., the subject's neural performance level, the subject's behavioral performance level or a combination thereof. For example, when the cognitive task is presented as part of a video game, feedback in the form of an in-game reward (e.g., bonus points) or penalty, such as a graphical or auditory representation thereof, may be provided to the subject upon the subject performing at a specified level. For example, positive feedback may be provided for improving behavioral performance, e.g., surpassing a particular score/number of points, “passing a level”, and the like. In some instances, positive feedback may be provided for improving neural performance, e.g., increased neural activity in a desired brain region, etc.

According to certain embodiments, the feedback may or may not be “tethered” to the subject's performance across both neural activity performance and behavioral performance. For example, in certain aspects, the methods of the present disclosure include rewarding a subject only when a threshold level of neural activity performance is achieved and a threshold level of behavioral performance is achieved. In certain aspects, the feedback may be tethered to only one component of performance. For example, in certain aspects, the methods of the present disclosure include rewarding a subject when a threshold of neural activity performance is achieved regardless of performance in other components, including e.g., behavioral performance.

In some embodiments, neural feedback may be part of a feedback loop that influences subsequent rounds of the cognitive task including whether the cognitive task increases or decreases in difficulty. Feedback loops utilized in the instant methods may be “open feedback loops” or “closed feedback loops” and, where a feedback loop influences subsequent rounds of the cognitive task, feedback loops may be adaptive open feedback loops or adaptive closed feedback loops.

In open feedback loops, with regards to neural activity, participants in a cognitive task respond to stimuli presented in the task, but real-time performance feedback of neural activity is not provided that in turn can modulate user responses on subsequent trials, nor are they adaptive to current user performance. For example, in an open feedback loop a subject may receive feedback based on performance of the cognitive task but irrespective of or unassociated with neural activity. As such, in certain open feedback loops a subject may receive positive feedback, e.g., in response to completing the cognitive task correctly, even when neural activity performance worsens or is below a desired level. Correspondingly, a subject may receive negative feedback, e.g., in response to an incorrect response on the cognitive task, even when neural activity performance improves or is above a desired level.

In a closed feedback loop, with regards to cognitive fitness and neural activity, participants in a cognitive task respond to stimuli presented in the task and real-time performance feedback of neural activity is provided that in turn can modulate user responses on subsequent trials. In addition, the presented cognitive task in closed feedback loop is adaptive to current user performance.

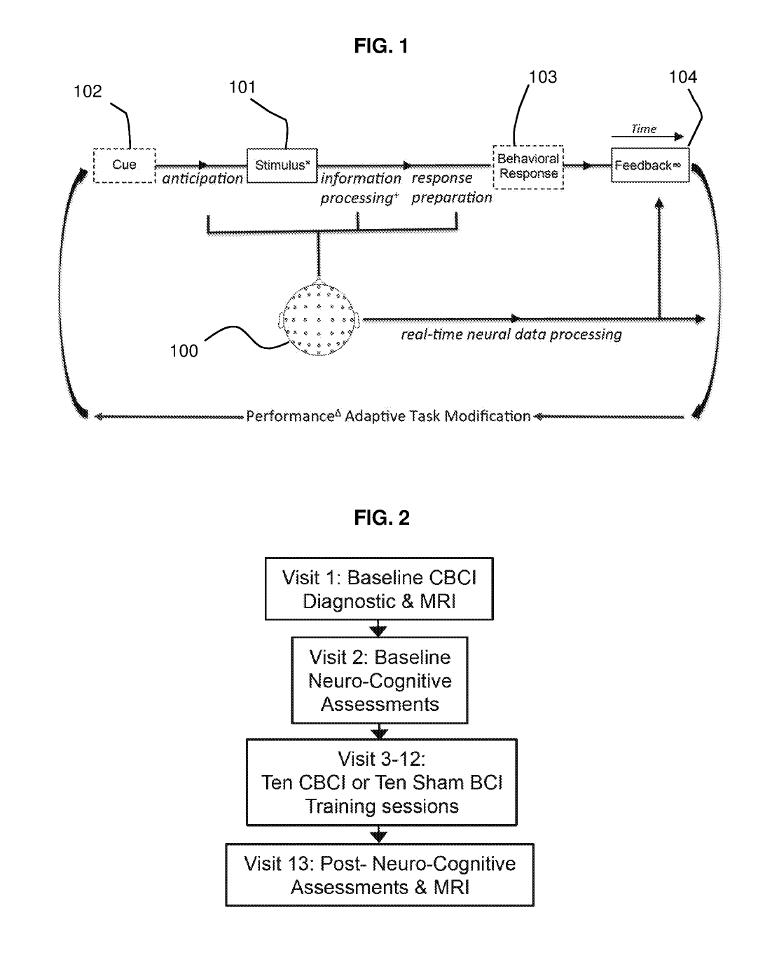

One embodiment of a closed feedback loop is provided in FIG. 1. As shown in the figure, a subject being monitored via a multi-channel EEG (100) is presented with a stimulus (101), with or without a prior cue (102) and a period of stimulus anticipation. Following the presentation of the stimulus the subject responds with an information processing event, a response preparation event and, optionally, a behavioral response event (103). Neural activity during one or more of such events, including the optional stimulus anticipation period and behavioral events, is detected and processed in real-time. The processed neural activity, representative of a neural performance level, is provided as feedback (104) into the closed loop which influences the subsequent task to be presented to the subject thus resulting in performance-based adaptive task modification. The closed loop cycles repeatedly such that each time the task is performed the neural performance feedback adaptively modifies the subsequent task presented to the subject. Feedback in such a loop may be based solely on the neural performance level or a combination of the neural performance level and some other factor including, e.g., behavioral performance level.

### Cognitive Training

In certain aspects, the cognitive task may be presented to the subject as part of a cognitive training program. According to certain embodiments, the training program includes presenting 2 or more, 3 or more, 4 or more, 5 or more, 10 or more, 15 or more, 20 or more, 25 or more, 30 or more, 35 or more, 40 or more, 45 or more, 50 or more, 55 or more, 60 or more, 65 or more, 70 or more, 75 or more, 80 or more, 85 or more, 90 or more, 95 or more, or 100 or more rounds of cognitive tasks of a selected duration, the difficulty levels of which may, in certain aspects, be adapted based on the subject's neural activity performance during presentation of a preceding cognitive task at a particular difficulty level. Such a training program may include presenting one or more cognitive tasks to a subject each day over a number of days (e.g., a number of consecutive or non-consecutive days), such that the training program includes multiple “sessions” where the one or more presentations of a cognitive task during a day constitutes a single session. The training program may be presented for any number of days, e.g., until a desired level of cognitive fitness is achieved. In certain aspects, the entire training program is presented on a single day. In other aspects, the entire training program lasts from 2 to 7 days, from 8 to 14 days, from 15 to 21 days, from 22 to 28 days, or any other number of days suitable for achieving a desired result.

The methods of the present disclosure may include a step prior to the first presenting step. For example, where the method is carried out for the first time for a subject, the method may encompass a thresholding step, an assessment, instruction, and/or demonstration. Such prior steps may, in some instances, be useful for tailoring a cognitive training program to an individual subject. For example, baseline assessments may serve to adapt the starting level of cognitive training to the stating cognitive ability of the subject.

A thresholding step includes presenting to a subject a cognitive task in one or more trials. This thresholding step helps determine a “baseline” performance level of the subject on the cognitive task. Another purpose of the thresholding is to determine a difficulty level to carry out an assessment. Thresholding to a specific difficulty level can be useful in tailoring the methods to an individual because each subject can have variable baseline abilities to perform the cognitive task (e.g., older adults perform certain tasks at a lower performance level when the task is matched in difficulty).

A difficulty level to carry out an assessment may be a level at which the subject performs the task with a pre-determined percentage of accuracy (e.g., 80%), optionally within a pre-selected maximum response time. The initial difficulty level may be a default difficulty level for a category of subjects (e.g. average for an age range), a lowest level of difficulty, or a level comparable based on the subject's prior assessment. The difficulty levels can then be adapted dynamically to one or more performance levels of the subject, including e.g., the neural performance level of the subject. Adaptation of the cognitive task may be performed according to a variety of methods including but not limited to e.g., staircase algorithm adaptation.

The threshold level can be personalized to the subject. For example, in certain aspects, a threshold level for the first time is a level at which the subject can perform the cognitive task with an accuracy of about 50% or more, about 55% or more, about 60% or more, about 65% or more, about 70% or more, about 75% or more, about 80% or more, about 85% or more, or about 90% or more. After a thresholding step, the methods may include an assessment before, during and/or after a training session or training program.

In some instances, the threshold level may be based on an observed neural activity including but not limited to e.g., an observed neural activity in a desired brain region, an observed neural activity of a desired magnitude, etc. For example, in the first and/or subsequent trial the threshold may be set at a level at which the subject displays a desired neural activity for about 50% or more of the trial, about 55% or more of the trial, about 60% or more of the trial, about 65% or more of the trial, about 70% or more of the trial, about 75% or more of the trial, about 80% or more of the trial, about 85% or more of the trial, or about 90% or more of the trial, etc.

As summarized above, according to certain embodiments, the methods of the present disclosure improve cognitive fitness in the subject. By “improve cognitive fitness” is meant that the subject's cognition is enhanced, i.e., at least one aspect of the subject's cognition is improved as a result of the method (or two or more iterations of the steps of the method, e.g., as part of a training session or training program). Aspects of the subject's cognition that may be enhanced include, but are not limited to, the subject's memory (e.g., working memory (e.g., working memory fidelity without interference, working memory fidelity with interference, working memory span with or without interference)), attention (e.g., sustained attention, response inhibition, attention regulation (e.g., with or without distractions (visual distractions, auditory distractions, emotional distractions, etc.)), task-switching ability, goal management ability, target search ability, target discrimination ability, and/or the like. Such improvements in cognitive fitness may also include improvements in neural activity of a desired level or in a desired neural pathway or both. In certain embodiments, improvements in neural activity may be present, at least initially, with or without measurable increases in task performance.

According to certain embodiments, improvement of the subject's cognitive fitness is determined by performing a pre-training assessment and a post-training assessment. The methods may also include one or more of the assessments intermittently throughout the training (e.g., inter-trial or inter-session). An assessment may include presenting a cognitive task and evaluating the performance of the subject, with or without a corresponding neural activity assessment. The assessment may be different from a training session in that it does not seek to train the subject. In one embodiment, unlike a training session, the difficulty level from trial to trial in an assessment does not change or adapt to the performance of the subject. Rather, the difficulty level for assessment purposes remains the same (e.g. at the difficulty level determined by a thresholding step). In another embodiment, the assessment does include an adaptation in difficulty level, e.g., by methods known in the art of psychometric analysis (e.g., staircase procedures and/or maximum likelihood procedures) to adaptively determine the ability of the subject. In either case, the primary purpose of the assessment is to evaluate the performance of the individual as opposed to train that performance.

The assessment described above can be conducted before and/or after a training session or training program. The steps involved in a post-training assessment are the same as those of a pre-training assessment described above, except that in a pre-training assessment, the data are used to determine the ability and/or performance of a subject prior to training. In a post-training session, however, the data analyzed may include data collected not just in the assessments but also during the training program. Additionally, the post-training assessment may be used as feedback to the subject, as well as feedback to the training program, as a control by which to direct the advancement of the next training session. Such post-training assessment feedback may be provided in addition to neural activity feedback provided according to the instantly described methods.

The analysis also reflects the performance and ability of the subject after training. In other words, post-training assessment can compare the performance of the subject post-training to that prior to training and assess the impact of training on the cognitive ability of the subject. When one or more cognitive abilities of the subject is improved post-training (as revealed by comparing the cognitive ability during a post-training cognitive ability assessment to the cognitive ability during a pre-training cognitive ability assessment), the subject's cognition has been enhanced by the method. Cognitive abilities that may be assessed include but are not limited to working memory, attention, task-switching, goal management, target search, target discrimination, and/or the like.

### Therapeutic Methods

As summarized above, included in the present disclosure are methods of cognitive training to enhance cognitive ability in a subject having cognitive dysfunction, thereby treating a cognitive disorder in a subject. The treatment methods include presenting to a subject having a cognitive disorder an adaptive cognitive task alone or as part of an adaptive cognitive training program. The adaptive component of the cognitive task may include adapting the difficulty level of each successive cognitive task based on the subject's performance on one or more of the preceding task where the performance assessed includes neural activity performance. The particular method steps may be performed using any of the approaches described herein.

Assessments of the treatment effects of an adaptive cognitive task and/or a training program utilizing an adaptive cognitive task may include one or more tests that measure improvement of symptoms or functions relevant to a specific disease or condition of the subject. Suitable types of tests include those that objectively measure symptom severity or biomarkers of a disease or condition, tests that use subjective clinician or observer measurement of symptom severity, and tests that measure cognitive functions known to be correlated with disease states. Examples of such tests include but are not limited to assessment scales or surveys such as the Mini Mental State Exam, CANTAB cognitive battery, Repeatable Battery for the Assessment of Neuropsychological Status, Clinical Global Impression scales relevant to specific conditions, Clinician's interview-Based Impression of Change, Severe Impairment Battery, Alzheimer's Disease Assessment Scale, Positive and Negative Syndrome Scale, Schizophrenia Cognition Rating Scale, Conners Adult ADHD Rating Scales, Hamilton Rating Scale for Depression, Hamilton Anxiety Scale, Montgomery-Asberg Depressing Rating scale, Young Mania Rating Scale, Children's Depression Rating Scale, Penn State Worry Questionnaire, Hospital Anxiety and Depression Scale, Aberrant Behavior Checklist, and Activities of Daily Living scales; physiological tests that measure internal markers of disease or health such as detection of amyloid beta, cortisol and other stress response markers; and brain imaging studies (for example fMRI, PET, etc.) that assess a condition based on presence of specific neural signatures.

Alternatively, or additionally, treatment assessment, including pre-training and post-training assessments, may include survey or questionnaire-style tests that measure a subject's self-reported perception of themselves. These can include self-report scales of healthy function or feelings, or disease function or symptoms. Examples of suitable self-report tests include but are not limited to ADHD self-report scale, Positive and Negative Affect Schedule, Depression Anxiety Stress Scales, Quick Inventory of Depressive Symptomatology, PTSD Checklist, and any other types of surveys that can be conducted for a subject to report on their general feelings of symptoms of a condition or satisfaction with real-world functional status or improvement.

Use of Methods in Conjunction with Other Therapeutics and Diagnostics

The methods described in the instant application can be used alone or with other interventions which are known to improve cognition and/or treat diseases and conditions. Other interventions include drugs as well as psychotherapeutic techniques. Sessions and training programs described herein may be used either consecutively or simultaneously with the other interventions. When used consecutively, the sessions and training programs can be used either prior to the other intervention or after the other intervention. The sessions and training programs may be used with one or multiple interventions.

Drug therapies that could be used in combination with the herein described methods or systems include, but are not limited to cholinesterase inhibitors, memantine, anti-depressants (e.g., selective serotonin-reuptake inhibitors, norepinephrine reuptake inhibitors, monoamine oxidase inhibitors, etc.), anxiolytics (e.g., benzodiazepines, buspirone, barbiturates, etc.) and antipsychotics.

Psychotherapeutic techniques that could be used in combination with the herein described methods or systems include, but are not limited to, behavior therapy, psychodynamic therapy, psychoanalytic therapy, group therapy, family counseling, art therapy, music therapy, vocational therapy, humanistic therapy, existential therapy, transpersonal therapy, client-centered therapy (also called person-centered therapy), Gestalt therapy, biofeedback therapy, rational emotive behavioral therapy, reality therapy, response based therapy, Sandplay therapy, status dynamics therapy, hypnosis and validation therapy.

### Cognitive Fitness Detection Methods

The present disclosure provides methods for detecting cognitive fitness and/or deficits thereof. The methods may be used in detecting a cognitive fitness deficit in a subject that is characteristic of a subject having a particular condition (e.g., a particular cognitive disorder), detecting the onset of a particular condition in a subject, detecting a change in a condition of the subject based on detecting a change in the cognitive deficit, detecting the absence of a cognitive fitness deficit (i.e., detecting cognitive fitness), detecting that a subject no longer has a particular cognitive fitness deficit characteristic of a condition, or any combinations thereof.

A cognitive fitness assessment of the instant disclosure, e.g., for use in detecting a normal or abnormal level of cognitive fitness, may or may not be performed as a loop. For example, in some instances cognitive fitness detection may be performed utilizing a single round of cognitive testing without repeating or looping the cognitive testing, which may include one or more cognitive tasks. As such, in some instances a single round of cognitive testing is sufficient to make a neural activity determination sufficient to detect a targeted level of cognitive fitness.

In some instances, looping of the cognitive testing regime is used in a method of cognitive fitness detection. For example, a determination of cognitive fitness may be made based on the progression of neural activity in successive rounds of a looped cognitive testing scheme. Progression of neural activity enhancement, in an adaptive closed loop, at a rate that exceeds a threshold may be indicative of cognitive fitness whereas progression of neural activity enhancement at a rate that does not exceed a threshold is indicative of substandard cognitive fitness.

Accordingly, depending on the particular context, cognitive fitness detection (e.g., detection of standard, above standard or substandard cognitive fitness) may be performed utilizing a single round or multiple rounds, including looped, cognitive testing having a neural activity component. Deficits in the neural activity performance in a cognitive assessment as described herein may indicate the presence of one or more cognitive deficits or conditions.

### Systems, Computer Readable Media and Computing Devices

Aspects of the present disclosure further include systems, computer readable media and computing devices, including where such systems, computer readable media and computing devices are configured to perform all or a part of any of the methods as described herein.

Systems of the instant disclosure will generally include a neural activity detector for detecting the neural activity of a subject before, during or after the presentation of a stimulus of a cognitive task to the subject. Useful neural activity detectors may vary widely and the selection of a particular detector in a subject system may depend, at least in part, on the required resolution of neural activity. As such, depending on the circumstances, useful neural activity detectors include but are not limited to e.g., an electroencephalogram (EEG) device, a functional magnetic resonance imaging (fMRI) device, a near-infrared spectroscopy (NIRS) device, an electrocortocography (ECoG) device, and combinations thereof.

In some embodiments, a neural activity detector of a subject system will generally be connected to, or have as one of its components, a data processing unit. Such data processing units may, e.g., convert the electrical signals of a neural activity detector into a recognizable form consisting of a physical representation of the neural activity in space and time. For example, in some instances, useful data processing units may convert the electrical signals represented neural activity to visible representations of the neural activity co-registered on an image of the brain of a subject or a generalize reference brain image. Accordingly, a data processing unit connected to or contained within a neural activity detector may include non-transitory programming containing instructions for the conversion of electrical signals from the neural activity detector into a temporal and/or spacial representation of the neural activity within a subject's brain.

The data generated from a neural activity detector of a subject system may be, with or without the use of a intervening data processing unit as described above, fed into a computing device configured to analyze the neural activity and adaptively modify the next and/or subsequently presented cognitive task(s). Accordingly, a neural activity detector of a subject system may be directly or indirectly (e.g., through an intervening data processing unit) connected to a centralized computing device for receiving the neural activity signals and modifying electrical control over attached components based on the received neural activity signals or processed data thereof.

In some instances, such a centralized computing device may serve as the basis for control of the entire system and thus may include additional attached components with various functions including but not limited to e.g., functions for controlling the presentation of a stimulus, functions for controlling the presentation of a cue, functions for receiving electrical signals from a neural activity detector, functions for processing electrical signals from a neural activity detector, functions for mapping or co-registering electrical signals from a neural activity detector onto a subject's brain image, functions for receiving a brain image of a subject, functions for measuring the strength of electrical signals from a neural activity detector, functions for comparing the strengths of electrical signals from a neural activity detector over time, functions for comparing the strengths of spatially separated electrical signals from a neural activity, functions for computing the neural performance level of a subject for one or more brain regions and/or neural pathways, functions for controlling the presentation of feedback to a subject undergoing a cognitive task, and the like. Such functions may be configured in hardware or software components and combinations thereof. For example, in some instances, a hardware component of the computing device provides for a particular function of the system. In some instances, software containing instructions may provide for a particular function of the system where such software may be stored on a computer-readable medium permanently or removeably attached to the computing device.

Components that may be attached to a computing device of a system for performing one or more of the methods described herein may include a user interface. A user interface of a subject system may serve various purposes including but not limited to presentation of a stimulus to a subject and/or presentation of feedback to the subject. Useful user interfaces for presenting a stimulus to a subject include but are not limited to e.g., a display (e.g., a series of indicator lights, a monitor, a projector, a virtual reality headset, etc.), an auditory device (e.g., a buzzer, a speaker, headphones, etc.), a tactile stimulator (e.g., a vibration device, a probe, etc.), an olfactory stimulator (e.g., a smell generator), a taste stimulator (e.g., a liquid and/or food dispenser, etc.). Such components of a user interface may be communicably connected, either unidirectionally connected or bidirectionally connected, to a computing device of the system by wired or wireless means.

In some instances, a user interface of a system as described herein may include one or more components for user input. For example, in some instances where the method includes a behavioural input from the subject, a system of the instant disclosure may include a user input with which the user provides the behavioural input. In some embodiments, the user input device may include a joystick, a controller, a steering wheel, a lever, a button, a touchscreen, a keyboard, a gamepad, a mouse, a trackball, a stylus, a wand, a gun, a knife, a handheld device, a wearable device, a biometric device, and the like. User input devices are not limited to tactile input and may in some instances include user input devices for auditory input (e.g., a microphone).

Components that may be attached to a computing device of a system for performing one or more of the methods described herein may include a brain imaging device. Any convenient brain imaging device may find use in the herein described systems including but not limited to e.g., a MRI scanner, a fMRI scanner, a Computed Tomography (CT) scanner, a positron emission tomography (PET) scanner, a Diffuse optical imaging (DOI) device, a Single-photon emission computed tomography (SPECT) device, a cranial ultrasound device, combinations thereof and the like.

In certain aspects, provided are non-transitory computer readable media including instructions stored thereon for causing a computer device/system to implement the methods of the present disclosure, including any embodiments of the methods described elsewhere herein. For example, the computer readable medium may include instructions to cause the computer device/system to present a stimulus to a subject through a user interface, present a cue to a subject through a user interface, receive electrical signals from a neural activity detector, time-lock detected neural activity to a stimulus-related event, map received electrical signals representing neural activity to a brain image, measure the strength of electrical signals representing neural activity, identify the location of electrical signals representing neural activity, compare the strength of electrical signals representing neural activity, compare the location of electrical signals representing neural activity, determine the neural performance level of a subject based on electrical signals representing neural activity, provide feedback to a subject, adapt a cognitive task based on electrical signals representing neural activity, present an adapted task to a subject, and the like.

Non-transitory physical computer readable media of the present disclosure include, but are not limited to, disks (e.g., magnetic or optical disks), solid-state storage drives, cards, tapes, drums, punched cards, barcodes, and magnetic ink characters and other physical medium that may be used for storing representations, instructions, and/or the like.

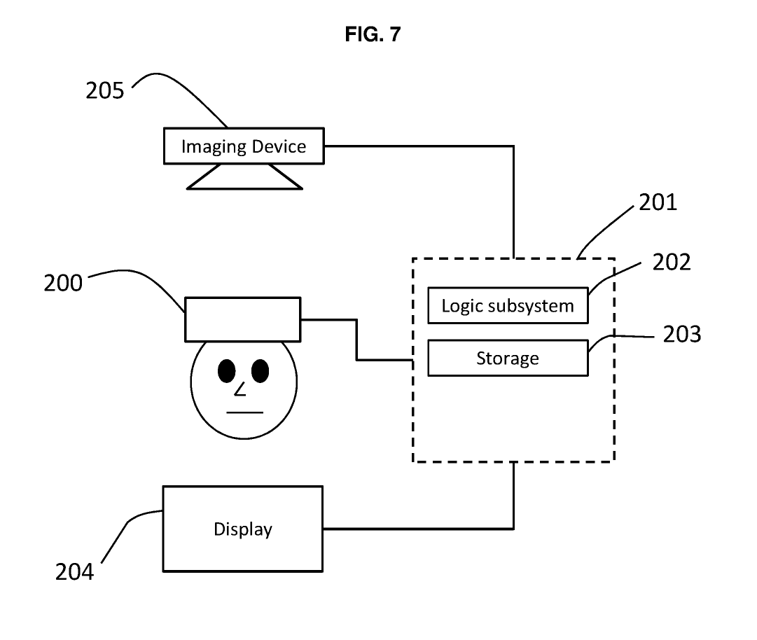

Referring now to the embodiment presented in FIG. 7, a system of the instant disclosure may include a neural activity detector (200) electrically connected, either wired or wirelessly, to a computing device (201) having a logic subsystem (202). The logic subsystem may include one or more processors configured to execute software instructions. Additionally or alternatively, the logic subsystem may include one or more hardware or firmware logic machines configured to execute hardware or firmware instructions. The processors of the logic subsystem may be single-core or multi-core, and the programs executed thereon may be configured for sequential, parallel or distributed processing. The logic subsystem may include individual components that are distributed among two or more devices, which can be remotely located and/or configured for coordinated processing. Aspects of the logic subsystem may be virtualized and executed by remotely accessible networked computing devices configured in a cloud-computing configuration.

The system may further include a storage subsystem (203) that includes one or more physical, non-transitory, devices configured to hold data and/or instructions executable by the logic subsystem to implement the methods of the present disclosure. When such methods and processes are implemented, the state of storage subsystem may be transformed, e.g., to hold different data.

The storage subsystem (203) may include removable media and/or built-in devices. Storage subsystems may include optical memory devices (e.g., CD, DVD, HD-DVD, Blu-Ray Disc, etc.), semiconductor memory devices (e.g., RAM, EPROM, EEPROM, etc.) and/or magnetic memory devices (e.g., hard-disk drive, floppy-disk drive, tape drive, MRAM, etc.), among others. Storage subsystems may include volatile, nonvolatile, dynamic, static, read/write, read-only, random-access, sequential-access, location-addressable, file-addressable, and/or content-addressable devices.

Storage subsystems may include one or more physical, non-transitory devices. However, in some embodiments, aspects of the instructions described herein may be propagated in a transitory fashion by a pure signal, e.g., an electromagnetic or optical signal, etc. that is not held by a physical device for a finite duration. Furthermore, data and/or other forms of information pertaining to the present disclosure may be propagated by a pure signal.

According to certain embodiments, aspects of logic subsystem and of the storage subsystem may be integrated together into one or more hardware-logic components. Such hardware-logic components may include field-programmable gate arrays (FPGAs), program-and application-specific integrated circuits (PASIC/ASICs), program- and application-specific standard products (PSSP/ASSPs), system-on-a-chip (SOC) systems, and complex programmable logic devices (CPLDs), for example.

The system may further include a user interface display component (204) used to present a stimulus of a cognitive task present in instructions held by storage subsystem to the subject. The cognitive task may take the form of a video game, a graphical user interface (GUI), and the like. As the methods change the data held by the storage subsystem, and thus transform the state of the storage subsystem, the state of the display component may likewise be transformed to visually represent changes in the underlying data. The display component may include one or more display devices utilizing virtually any type of technology. Such display devices may be combined with the logic subsystem and/or the storage subsystem in a shared enclosure, or such display devices may be peripheral display devices such as the display component shown in FIG. 7.

The system may optionally include a brain imager (205) connected, wired or wirelessly, to the computing device. As described above, in embodiments of the system that include a brain imager, any convenient brain imaging device may be employed. In some instances, the neural activity detector (200) and the brain imager (205) may be one and the same device including e.g., where the neural activity detector and the brain imager are both an fMRI.

### Utility

The methods of the present disclosure find use a variety of contexts, and in certain instances, provide for the detection of cognitive defects, the enhancement of cognitive function in healthy individuals, the treatment of cognitive dysfunction in subjects having a condition in need thereof, and the like.

Individuals that can use the methods and tools of the invention can be any person, including those interested in enhancing cognitive abilities including those with normal cognitive ability, those with impaired cognitive ability and those at risk of impaired cognitive ability. Accordingly, there are many potential populations that would benefit from the new training methods as described herein.

Individuals that can benefit from the subject methods and tools include but are not limited to adults, such as aging adults. For example, the subject methods and systems can be useful for adults that are of any age. It is well-known that many healthy aging adults have a significant deficit in certain cognitive abilities. Such decline typically accelerates at age 50 and older and over subsequent decades, such that these lapses become noticeably more frequent. It is often clinically referred to as “age-related cognitive decline”. While often viewed (especially against more serious illnesses, such as Alzheimer's disease, Parkinson's disease) as benign, such predictable age-related cognitive decline can severely alter quality of life by making daily tasks arduous.

Age-related cognitive decline can lead to a more severe condition now known as Mild Cognitive Impairment (MCI), in which sufferers show specific sharp declines in cognitive function relative to their historical lifetime abilities while not meeting the formal clinical criteria for dementia. The subject methods and systems have the potential to reverse and/or prevent the onset of this devastating neurological disorder in humans, such as those suffering or at risk for MCI.

Aside from age-related cognitive decline, people of all ages who experience or are at risk for cognitive impairment can benefit the methods and systems of the present disclosure. For example, the present methods and systems are useful for training individuals whose cognitive losses have arisen as a consequence of injury (e.g., traumatic brain injury), medical treatments, chronic neurological, psychiatric illness, or of unknown cause. Such cognitive impairment, age-related or not, can be a contributing factor or manifesting symptom of a variety of conditions, including Alzheimer's disease, Parkinson's disease, Huntington's disease, depression, schizophrenia, dementia (including, but not limited to, AIDS related dementia, vascular dementia, age-related dementia, dementia associated with Lewy bodies and idiopathic dementia), Pick's disease, cognitive deficit associated with fatigue, multiple sclerosis, post-traumatic stress disorder (PTSD), obsessive-compulsive disorder (OCD), and others. Other cognitive losses can include brain damage attributable to infectious pathogens, medical intervention, alcohol or drugs, etc. Thus, cognitive decline or impairment can be a contributing factor or negative influence on a variety of adverse conditions, and thus the present invention can be useful in combating or diagnosing anxiety, stress, panic, depression, dysphoria, or malaise. Additionally, cognitive decline may result as a secondary symptom from a variety of disease states that are on the surface unrelated to cognition, but which significantly adversely affect the above-mentioned cognitive processes. Accordingly, individuals experiencing pain or diseases having a significant pain component, insomnia, or adverse effects of disease treatment such as chemotherapy or radiation therapy can also find use in methods of the present disclosure.

Populations that can benefit from the present methods further encompass those that suffer from attention deficit disorder (e.g. attention deficit hyperactivity disorder (ADHD)). Cognitive losses of developmentally impaired child and adult populations, encompassing general or undiagnosed developmental delays, Autism Spectrum Disorders (ASDs) (e.g. Aspberger's), can also be potentially reversed by the subject method.

Additionally, many individuals, though not experiencing a perceptible decline in cognitive function, may desire to increase their current cognitive abilities. One example is to improve the performance of everyday tasks (e.g. multitasking, focus, memory, social skills, such as conversational skills, decision-making abilities, creativity, or reaction times to specific task). Another example is to improve general metrics of cognitive ability (e.g. to “enhance IQ”). Secondary effects dependent on the above mentioned and trained cognitive abilities may also be a target for training using the present invention.

Given the diversity of subjects, both healthy and impaired, for which the instant methods and systems may provide a benefit, in many instances the subject of the described method is a human subject, e.g., a female or male human subject, of various ages. Thus, human subjects of interest include children and adults. In certain aspects, the human subject is from 4 years old to 100 years old, such as from 8 years old to 100 years old, from 9 years old up to 90 years old, from 10 years old up to 80 years old, from 11 years old up to 75 years old, or from 12 years old up to 70 years old. According to certain embodiments, the human subject is a child (newborn up to 18 years old). Children of interest include infants (newborn up to 1 year old), toddlers (1 year old up to 3 years old), preschoolers (3 years old up to 4 years old), children in middle childhood (4 years old up to 11 years old, such as 6 years old up to 8 years old, or 8 years old up to 11 years old), young teens (11 years old up to 14 years old) and teenagers (14 years old up to 18 years old). When the subject is a human adult, the subject may be a younger adult (an 18-30 year-old adult, e.g., a 21-28 year-old adult)), a middle-age adult (a 31-49 year-old adult), or an older adult (a 50 year-old or older adult (e.g., a 57-75 year-old adult)).

The subject may have a cognitive disorder, or the subject may be a healthy subject. As used herein, a “cognitive disorder” is a disorder that affects one or more mental processes, including impairments in one or more aspects of cognitive control, such as memory (e.g., working memory), attention, task-switching, goal management, target search, target discrimination, self-regulation, language comprehension and emotional processing. Such disorders may be accompanied by personality and behavioral changes. A “healthy subject” is a subject who does not have a cognitive disorder.

The following examples are offered by way of illustration and not by way of limitation.

### EXPERIMENTAL

### Example 1

### A Closed Loop Neural Cognitive Brain Computer Interface

A Cognitive Brain Computer Interface (CBCI) was designed that can directly target the neural processes underlying cognitive performance and integrate these with a computerized digital environment. CBCIs enable the user to digitally interact with their neural activity patterns underlying cognition, and intrinsically modulate these processes guided by digital neural feedback. Using a neural closed loop CBCI, cognitive task challenge can also be dynamically adapted based on neural performance.

The designed algorithmic pipeline records high-density (64-channel) electroencephalography (EEG) while the participant is engaged in a cognitive task. Then in real-time (i.e., with a minimal 100-200 millisecond time lag), the pipeline computes neural signals that are: (i) corrected for ocular and muscular artifacts resulting from eye movements, blinks and facial muscle movements, (ii) source localized to underlying cortical regions with high precision based on a head model that is co-registered to a previously acquired structural magnetic resonance image (MRI) in each participant, and (iii) decomposed into power and coherence measures in physiologically relevant frequency bands of theta (4-7 Hz), alpha (8-14 Hz) and beta (15-30 Hz).

The CBCI neural computations directly interface with a cognitive task, which is developed on the Unity 3D game engine such that the participant receives neural performance feedback on each trial of the task. Additionally, the parameters dictating task demands (trial time, stimulus durations, degree of task interference etc.) are adaptively modified based on current neural performance.

The neural signals that compose this measure are those that most robustly relate to on-task behavioral performance in a prior open loop ‘CBCI diagnostic’ experiment, described below. All participants in CBCI training undergo the CBCI diagnostic assessment, in which they perform the CBCI cognitive task in the absence of neural feedback or adaptivity. The neural signals that are most relevant to cognitive performance in the spatial-spectral-temporal domain are identified as those that differentiate correct and incorrect on-task behavior within each participant, and which also show significant neurobehavioral correlation across participants. Using a combined within- and across-participant analytical approach to find the neural performance signals in the diagnostic allows for integrating the same neural performance metric during the CBCI closed loop learning phase for all participants. Doing so demonstrates how targeting a specific neural process (or set of neural processes) in a closed loop induces neural plasticity in that process as well as how cognitive performance is influenced across several participants.

Within the CBCI closed loop, neural performance feedback is provided to the participant on each task trial on a personalized scale, whose range is determined by the mean and variance of their neural performance signal during the diagnostic test. Adaptive modifications to the task challenge also take place on a staircase scheme with a step-size scaled to the participant's personal neural performance ability. Of note, the custom-designed neural closed loops utilize personalized performance feedback and adaptive mechanics to effectively harness neuroplasticity and enhance cognition.

Thus, the CBCI neurotechnology provides novel real-time cognitive task based neural processing, cortical source-localized outputs using high-density EEG co-registered to structural MRI, and incorporation of real-time feedback and performance adaptive mechanics.

### Example 2

### Trial of Closed Loop Neural CBCI Cognitive Training

A study was designed to test the cognitive training efficiency of the closed loop neural CBCI described above using healthy, screened human voluntary participants. The Experimental Flow is outlined in FIG. 2. Essentially, on the first baseline visit, participants perform the open loop CBCI diagnostic assessment and also undergo an MRI scan. This assessment (detailed in the CBCI Diagnostic, below) serves three purposes: behavioral thresholding, acquisition of EEG and MRI neural parameters for subsequent closed loop personalization, and confirmation of the neural performance measure in each study participant.

On the second visit, participants undergo baseline assessments on a battery of neuro-cognitive function tests. The cognitive tests in this battery have been standardized over several hundred participants and hence have robust validity and reliability. All cognitive tests in the battery are accompanied by simultaneous EEG recordings to document the neural measures underlying cognitive performance, and to track how these neural and cognitive performance outcomes change post-CBCI-learning.

On the third through twelfth visits, participants are randomized to a CBCI learning or a sham BCI study arm, and all participants engage in their respective study arms for ten in-lab sessions. The duration of each learning session is 40 minutes, and the ten sessions are performed at an average frequency of 3-5 sessions per week, with training completed in 3-5 weeks.

Both CBCI learning and control (sham BCI) groups in the study are blind to their arm assignment. The sham BCI group is matched for practice and placebo effects. The sham BCI learning sessions appear identical to the ones in the CBCI learning group, except that the neural closed loop for sham participants does not incorporate personal neural performance metrics, but is instead yoked to trial-by-trial neural data from age- and gender-matched participants in the CBCI group. Using yoked neural signals in the sham group, instead of random neural signals, ensures that the sham BCI group participants receive positive/negative neural feedback and neurally-adaptive task challenges in similar proportions as experienced by the CBCI group. Efficiency of cognitive training is tracked throughout all sessions, using detailed trial-by-trial measures of cognitive and neural performance for all participants.

On the final visit, participants undergo a post-training evaluation of their neuro-cognitive functions on the standard test battery used at baseline (visit 2). Again, EEG is recorded simultaneous to all tasks to analyze CBCI-learning related changes in cognitive and neural function. All participants repeat an MRI/functional MRI scan at this final session to uncover structural and functional neuroplastic changes related to CBCI learning.

### Example 3

### CBCI Diagnostic Testing, Recording and Metric Identification

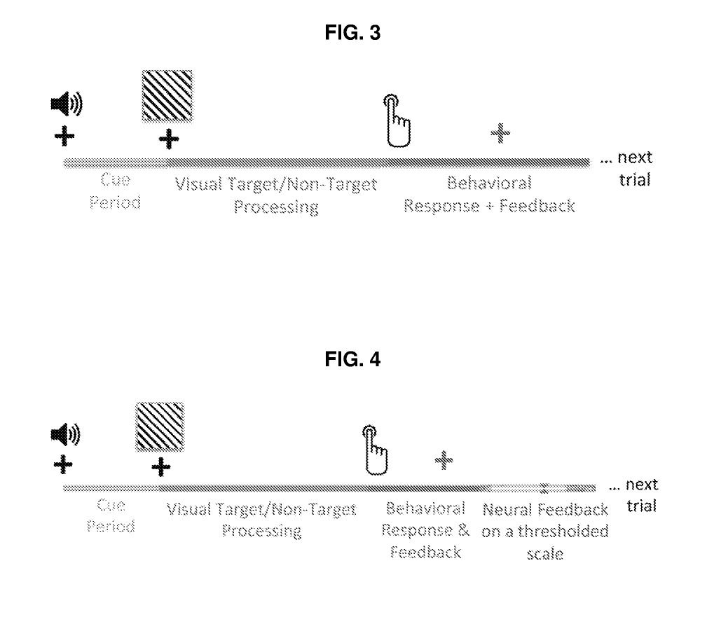

A CBCI diagnostic task was devised for use in CBCI cognitive fitness training, e.g., as a baseline as described above, and in CBCI neural diagnostics. The devised CBCI diagnostic task (FIG. 3) is a simple cued selective attention task. Task trials begin with an audiovisual cue (of 0.1 sec duration) alerting the participant to get ready for an upcoming visual stimulus. After a 1 sec cue period, a visual grating appears on the screen for 0.1 sec. The grating can be any one of five shapes (square, circle, diamond, pentagon, hexagon all of equal area) and can have any one of two orientations (45° or 135°). One of these stimuli is designated as the target stimulus of a specific shape and orientation combination and is pre-defined prior to starting the experimental run, while all other stimuli are non-targets. Targets occur infrequently on 33% of trials, with a new target defined for each of ten diagnostic runs.

Participants make a two-alternative forced choice response between one of two joystick response buttons assigned for the target vs. non-target stimuli. Participants then receive behavioral (but no neural) feedback (of 0.1 sec duration) on their performance: the fixation cross-hair turns green to indicate fast and accurate responding or turns red to indicate slow and/or incorrect responding. The threshold for fast vs. slow responding is user-specific and is determined using a staircase thresholding procedure on the first of ten diagnostic runs. The response threshold converges to a value at which participants have 80% response accuracy—a point at which they feel engaged and challenged but not frustrated. The diagnostic assessment lasts 30-40 min, with 10 experiment runs of 75 trials each (750 total trials) and short breaks in between to prevent fatigue.

The CBCI diagnostic records and processes the participant's EEG simultaneous to engagement in the cued selective attention task. EEG is acquired using the BioSemi Active Two 64-channel system with signals amplified and digitized at 1024 Hz with 24-bit resolution. Electrode positions are documented using the Brainsight® spatial digitizer and co-registered to each participant's MRI structural scan. The MRI scan is obtained on a Siemens 3T Trio Tim scanner with a 12-channel coil. High resolution T1-MPRAGE images are acquired for anatomical localization, normalization and use in morphometric analyses. MRI data processing uses standard Freesurfer tools, and EEG data is co-registered to the MRI anatomical reconstructions in each participant using cortically-constrained MNE source localization using the Brainstorm EEG/MRI processing toolkit. The cortical surface is divided into 68 anatomical regions of interest (ROIs; 34 in each hemisphere) based on the Desikan-Killiany atlas. The EEG data are then processed as event-related spectrally decomposed measures of neural activity, power and coherence, determined for these source-localized ROIs using inverse modeling.

A diagnostic study was performed to identify a neural performance metric. 40 healthy young participants were enrolled in a CBCI diagnostic study to ascertain the neural signatures that are associated with high cognitive performance. As high performance can be defined both in terms of high accuracy and fast response times (RT), a cognitive efficiency metric was adopted that accounts for both accuracy and RT variables. For this, each correct task trial was scored as 1/RT and incorrect trial as 0, and the average cognitive efficiency calculated for each participant. Complementary across- and within-participant analyses were then performed to identify the neural signatures that underlie high cognitive performance, which are then be used as a neural performance metric in a CBCI neural closed loop.

Across participants, regression analyses were performed for average cognitive efficiency versus average neural measurements of power and coherence in the theta, alpha and beta bands within the 0-1.5 sec trial interval from cue onset until the average participant response time. Within participants, the power and coherence neural measures on correct and fast trials (i.e. within the participant's RT threshold) vs. incorrect trials were compared. Then, to ascertain the neural measures that reliably predict cognitive performance across- and within-participants, their intersection was calculated, i.e. neural measures that are significant in both analyses were focused on. To eliminate intersections between the two analyses that can occur by random chance, the intersection matrices were bootstrapped (at p<0.01 or >99.5% CI) in each frequency band. Only the coherence, but not power, analyses survived the bootstrapping, and the highest proportion of significant intersections was found in the alpha band (proportion of significant intersections: 32% theta, 45% alpha and 23% beta). Hence, further analyses focused on the alpha coherence effects, and to estimate which functional connections best predict cognitive efficiency, these outcomes were subjected to a stepwise regression model.

It was found that early anticipatory alpha coherence (0-0.5 sec post-cue) between left prefrontal (caudal middle frontal region) and left extrastriate visual cortex (middle temporal, inferior temporal, fusiform complex) best predicted cognitive performance (R2=0.35, p<0.001). Specifically, lower alpha coherence of these prefrontal-sensory connections during early cue processing was associated with higher cognitive efficiency across participants. Further, within participant correct vs. incorrect trials significantly differed in this alpha coherence measure, with greater prefrontal-sensory alpha coherence on incorrect trials. Accordingly, this identified neural network was identified for specific targeting in real-time neuro-modulation of during closed loop CBCI learning.

### Example 4

### Adaptively Adjusted Closed Loop Neural CBCI Training

Neural CBCI closed loop cognitive training was performed on volunteer human subjects and compared to sham brain-computer interface (BCI) training. A CBCI diagnostic session was used to inform the CBCI training in several ways: (1) It confirmed the neural performance metric that is integrated in the closed loop training. (2) It determined the RT threshold for each participant, as their mean+sd RT across all diagnostic trials (sd: standard deviation) at which participants perform at near 80% accuracy. (3) It personalized the neural bounds for the modulation of the neural performance metric during training: a 0-100 neural performance scale was implemented during training whose mid-point, 50, is the mean neural performance signal (prefrontal-sensory alpha coherence) during the diagnostic, and 0 and 100 represent +2.5 sd and −2.5 sd of the neural performance signal. Note that the 0-100 scale tracks decreasing coherence values, i.e. +2.5 sd to −2.5 sd, as it was found that lower prefrontal-sensory alpha coherence is better for cognitive performance.

The CBCI closed loop of this assay is similar to the diagnostic task with the exception that participants now received neural performance feedback on each task trial (of 0.63 sec duration) subsequent to behavioral performance feedback (FIG. 4). Thus, the participant's goals were to correctly perform the cued visual discrimination task, and to achieve high neural performance scores by maintaining attention on task. The neural performance feedback represents the magnitude of their anticipatory prefrontal-visual alpha coherence, and was shown on each trial relative to a threshold for expected neural performance. If the participant managed to surpass the neural performance threshold (initiating at +1 sd alpha coherence obtained from the CBCI diagnostic), the threshold moved up; else if the expected neural threshold was not met for two trials in a row, the threshold was relaxed (1up-2down staircase). Thus, participants were adaptively driven towards higher neural performance on each trial, which translated to successful reduction in the targeted prefrontal-sensory alpha coherence.

On trials when participants were both behaviorally and neurally successful, i.e. made correct behavioral discriminations and were also able to exceed their neural performance threshold, they received a medal presented during the neural feedback period. The number of medals was tracked across every training run of 75 trials, and the next run presented an adaptively adjusted increase or decrease in task difficulty based on the previous run's proportion of medal-trials. Change in task difficulty was implemented by increasing or decreasing the interference of the non-target stimuli. Non-target stimuli were of high-interference when they shared a feature, either shape or orientation, with the target stimulus and were of low-interference if they didn't share any features with the target. The proportion of high vs. low interference non-targets was adaptively modified to increase or decrease the task challenge in the next run, while keeping the total number of non-targets constant. Overall, adaptive learning on the CBCI continues for ten (non-consecutive) training days over 3-5 weeks; and each day's initial adaptive parameters are updated based on performance on the previous training day, in terms of RT thresholds, neural performance thresholds and task challenge level.

The active control group also engages in similar durations of training as the CBCI group. The user-facing end of the training is identical for the two groups with identical task goals. Only the backend computations differ in that the neural closed loop in the sham BCI group is decoupled from the participant's neural activity, but instead is yoked to an age- and gender-matched participant in the CBCI group. This yoking ensures the same ratio of positive and negative neural feedback trials in both groups. But learning in the sham BCI group is not benefitted by personalized neural feedback and only occurs due to task repetition, which is largely ineffective in driving neuroplasticity in isolation.

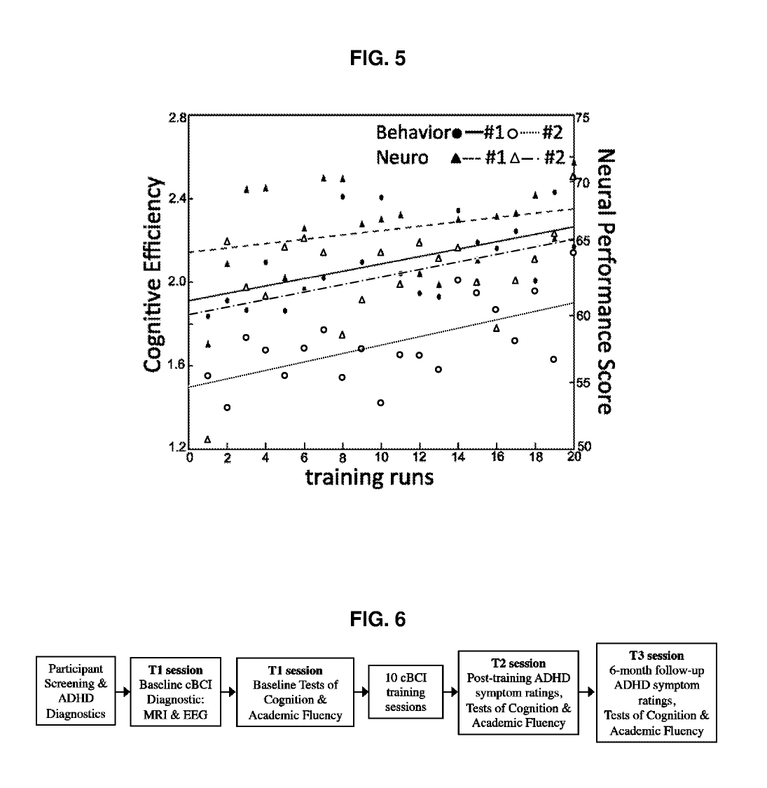

Tracking functionality of the CBCI closed loop was automated, evaluated through real-time trial-by-trial tracking of on-task behavioral and neural performance. FIG. 5 shows behavioral cognitive efficiency and neural performance score data from two participants (“#1” and “#2”) who underwent CBCI training sessions (20 training runs, 10 per session). Improvement trends were observed for both behavior (“Behavior”) and neural (“Neuro”) performance scores. Note that changes in the neural performance metric over the learning sessions represent plasticity in the underlying neural signal, i.e. anticipatory alpha coherence in prefrontal-visual cortical connections.

These data demonstrate the effectiveness of neural closed loop CBCI training, adaptive based on neural performance feedback, in improving both behavioral performance and neural performance in human subjects.

### Example 5

### Glass Brain Adaptive CBCI Training for Attention Deficit Hyperactivity Disorder (ADHD)

The “Glass Brain” is the most anatomically accurate 3D model of real-time human brain activity currently available. It integrates high spatial resolution brain structure information from magnetic resonance imaging (MRI) with high temporal (millisecond) resolution neural dynamics from electroencephalography (EEG).

Specifically, an MRI structural brain scan is used to build the high-resolution 3D anatomical head model and a DTI scan (Diffusion Tensor Imaging) reconstructs white matter tracts. High-density 64-channel EEG recordings, which measure neural activity over the entire scalp, are co-registered to the MRI-DTI head model of each individual. An algorithmic cBCI pipeline is then used to process real-time neural dynamics that can be visualized on the “Glass Brain” with a minimal 100-200 millisecond (msec) computation lag. These neural dynamics can be (1) simultaneously parsed into different physiologically relevant frequency bands (color-coded for 4-8Hz theta, 8-12 Hz alpha and 12-20 Hz beta), (2) are corrected for ocular & muscular artifacts, and (3) are localized to their neural cortical sources using inverse modeling. Real-time effective connectivity is also calculated as Granger-causal interactions and visualized as pulses of light flowing along the anatomical fiber tracts connecting brain regions.

Within the “Glass Brain” cBCI individuals engage in a challenging selective attention task, discriminating goal-relevant target information from distractions. Neural performance signals, specifically the communication between top-down prefrontal control sites and visual sensory brain regions, which are known to be a neurobiological correlate of attention regulation, can be monitored in real-time. When applied to ADHD, the “Glass Brain” cBCI could allow users to improve their attention-related neural processing in a personalized and targeted manner.

On each task trial, the user receives feedback on the strength of their on-task neural performance, i.e. the strength of their prefrontal-sensory coherence (PSC). Digital feedback and cumulating rewards then drive the user to enhance their PSC neural performance over multiple sessions of training. The “Glass Brain” cBCI is adaptive to an individual's neural performance such that individuals are constantly challenged to improve upon the extent of their neural signal modulation. Thus, higher training levels are more difficult and demand greater neural modulation for the same reward than lower training levels. The adaptive mechanics are personalized to the user in that the exact neural performance demanded at each progressive step is normalized within the range of the individual's neural modulation capabilities.

A study is conducted as a single-arm open-label feasibility trial of cBCI in adolescent children with ADHD. Ten sessions of cBCI training distributed over a 5 week period is flanked by baseline (T1), post-assessment (T2) and 6-month follow-up (T3) assessments measuring cognitive function, ADHD symptoms and academic (reading & math) fluency (FIG. 6).

20 adolescents (12-16 years) with ADHD and no other co-morbid major psychiatric disorder, as determined in an initial screening visit, are recruited for the study. At the T1 assessment visit, participants perform a 1-hour cognitive test battery and academic fluency tests, and provide ADHD self & observer behavior ratings. Then participants engage in ten 1-hour “Glass Brain” cBCI neurofeedback sessions in the lab over 5 weeks. With cBCI training, participants learn to improve their neural performance underlying selective attention. T2 (post-cBCI) and T3 (6-month follow-up) assessments are identical to the T1 visit. Statistical data analyses evaluate significant change in cognitive tests, ADHD symptoms and academic fluency at T2 and T3 relative to the T1 baseline session. Correlation analyses assess the relationship between neural and behavioral (cognitive, clinical and academic) improvements.

Notwithstanding the appended claims, the disclosure is also defined by the following embodiments:
1. A method comprising:
   presenting a cognitive task to a subject, wherein presenting the cognitive task comprises presenting a stimulus or sequence of stimuli to the subject;
   monitoring neural activity of the subject during the presenting of the cognitive task, wherein the neural activity comprises neural activity underlying one or more stimulus-related events, and the monitoring is time-locked to the one or more stimulus-related events;
   determining a neural performance level of the subject based on the neural activity underlying the one or more stimulus-related events; and
   adapting the cognitive task based on the neural performance level.
2. The method according to Embodiment 1, wherein the one or more stimulus-related events comprises information processing, the information processing comprising cognitive processing and sensory processing.
3. The method according to Embodiment 2, wherein determining a neural performance level of the subject is based on neural activity underlying the cognitive processing, the sensory processing, or both.
4. The method according to Embodiment 3, wherein adapting the cognitive task based on the neural performance level comprises adapting an aspect of the cognitive task relating to cognitive processing, sensory processing, or both.
5. The method according to any one of Embodiments 1 to 4, wherein presenting the cognitive task comprises presenting a cue prior to presenting the stimulus or sequence of stimuli to the subject.
6. The method according to Embodiment 5, wherein the one or more stimulus-related events comprises stimulus anticipation.
7. The method according to Embodiment 6, wherein determining a neural performance level of the subject is based on neural activity underlying the stimulus anticipation.
8. The method according to Embodiment 7, wherein adapting the cognitive task based on the neural performance level comprises adapting an aspect of the cognitive task relating to stimulus anticipation.
9. The method according to any one of Embodiments 1 to 8, wherein the cognitive task requires the subject to respond to the stimulus.
10. The method according to Embodiment 9, wherein the one or more stimulus-related events comprises response preparation.
11. The method according to Embodiment 10, wherein determining a neural performance level of the subject is based on neural activity underlying the response preparation.
12. The method according to Embodiment 11, wherein adapting the cognitive task based on the neural performance level comprises adapting an aspect of the cognitive task relating to response preparation.
13. The method according to any one of Embodiments 1 to 12, wherein the cognitive task targets an aspect of cognition selected from the group consisting of: attention, working memory, task-switching, goal management, target search, target discrimination, and any combination thereof.
14. The method according to Embodiment 13, wherein the cognitive task is an attention task.
15. The method according to Embodiment 14, wherein the attention task is a selective attention task.
16. The method according to Embodiment 15, wherein the selective attention task requires the subject to discriminate target information from distractions.
17. The method according to any one of Embodiments 1 to 16, wherein the stimulus or sequence of stimuli comprises a visual stimulus, an auditory stimulus, a tactile stimulus, an olfactory stimulus, or any combination thereof.
18. The method according to any one of Embodiments 1 to 17, wherein the monitoring comprises measuring neural activity of the subject by electroencephalography (EEG), functional magnetic resonance imaging (fMRI), near-infrared spectroscopy (NIRS), electrocortocography (ECoG), or a combination thereof, as the subject performs the cognitive task.
19. The method according to Embodiment 18, wherein the monitoring comprises co-registering the neural activity of the subject with a 3-dimensional (3D) structural model of the subject's brain.
20. The method according to Embodiment 19, comprising producing the 3D model of the subject's brain by performing a magnetic resonance imaging (MRI) structural brain scan on the subject prior to or during the presenting of the cognitive task.
21. The method according to any one of Embodiments 1 to 20, comprising providing an indication to the subject of the subject's neural performance level.
22. The method according to Embodiment 21, wherein the indication comprises an award.
23. The method according to any one of Embodiments 1 to 22, wherein the subject has a cognitive deficit selected from the group consisting of: attention deficit hyperactivity disorder (ADHD), post-traumatic stress disorder (PTSD), major depressive disorder, dementia, or a combination thereof.
24. A system for neural activity detection and adaptive training, the system comprising:
   a user interface;
   a neural activity detector;
   a computing device comprising a non-transitory computer readable medium storing instructions that, when executed, cause the computing device to:
      present, through the user interface, a first cognitive task to a subject comprising a stimulus or sequence of stimuli to generate stimulus-related events in the brain of the subject;
      receive electrical signals from the neural activity detector during the presentation of the cognitive task that represents neural activity underlying the stimulus-related events in the brain of the subject;
      map the electrical signals in real-time onto a 3D model of the subject's brain to locate the neural activity;
      measure the strength of the located neural activity;
      determine a neural performance level of the subject based on the measured neural activity;
      present, through the user interface, a second cognitive task to the subject adapted according to the determined neural performance level.
25. The system according to Embodiment 24, wherein the user interface comprises a display device adapted to relay a visual stimulus of the first and second cognitive tasks to the subject.
26. The system according to Embodiment 24 or 25, wherein the user interface comprises an auditory device adapted to relay an audible stimulus of the first and second cognitive tasks to the subject.
27. The system according to any one of Embodiments 24 to 26, wherein the user interface comprises a tactile stimulator adapted to relay a tactile stimulus of the first and second cognitive tasks to the subject.
28. The system according to any one of Embodiments 24 to 28, wherein the user interface comprises an olfactory stimulator adapted to relay an olfactory stimulus of the first and second cognitive tasks to the subject.
29. The system according to any one of Embodiments 24 to 28, wherein the user interface comprises a taste stimulator adapted to relay a taste stimulus of the first and second cognitive tasks to the subject.
30. The system according to any one of Embodiments 24 to 29, wherein the neural activity detector comprises a device selected from the group consisting of: an electroencephalogram (EEG) device, a functional magnetic resonance imaging (fMRI) device, a near-infrared spectroscopy (NIRS) device, an electrocortocography (ECoG) device, and a combination thereof.
31. The system according to any one of Embodiments 24 to 30, wherein the 3D model of the subject's brain is generated from a magnetic resonance imaging (MRI) structural brain scan of the subject's brain.
32. The system according to Embodiment 31, wherein the system further comprises a MRI scanner and the non-transitory computer readable medium further stores instructions that, when executed, cause the computing device to trigger the MRI scanner to generate the MRI structural brain scan of the subject's brain.
33. The system according to any one of Embodiments 24 to 32, wherein the non-transitory computer readable medium further stores instructions that, when executed, cause the computing device to trigger the user interface to provide feedback to the subject based on the neural performance level of the subject.
34. The system according to any one of Embodiments 24 to 33, wherein the user interface further comprises a user input device adapted to allow the subject to input a behavioral response to the stimulus or sequence of stimuli.
35. The system according to any one of Embodiments 24 to 34, wherein the non-transitory computer readable medium further stores instructions that, when executed, cause the computing device to assess the subject's behavioral performance level on the cognitive tasks and adapt the cognitive task based on both the neural performance level and the behavioral performance level.
36. The system according to any one of Embodiments 24 to 35, wherein the non-transitory computer readable medium further stores instructions that, when executed, cause the computing device to trigger the user interface to provide feedback to the subject based on the behavioral performance level of the subject.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method comprising:
presenting a cognitive task to a subject, wherein presenting the cognitive task comprises presenting a stimulus or sequence of stimuli to the subject;
monitoring neural activity of the subject during the presenting of the cognitive task, wherein the neural activity comprises neural activity underlying one or more stimulus-related events, and the monitoring is time-locked to the one or more stimulus-related events;
determining a neural performance level of the subject based on the neural activity underlying the one or more stimulus-related events; and
adapting the cognitive task based on the neural performance level.

2. The method according to claim 1, wherein the one or more stimulus-related events comprises information processing, the information processing comprising cognitive processing and sensory processing.

3. The method according to claim 2, wherein determining a neural performance level of the subject is based on neural activity underlying the cognitive processing, the sensory processing, or both.

4. The method according to claim 3, wherein adapting the cognitive task based on the neural performance level comprises adapting an aspect of the cognitive task relating to cognitive processing, sensory processing, or both.

5. The method according to any one of claims 1 to 4, wherein presenting the cognitive task comprises presenting a cue prior to presenting the stimulus or sequence of stimuli to the subject.

6. The method according to claim 5, wherein the one or more stimulus-related events comprises stimulus anticipation.

7. The method according to claim 6, wherein determining a neural performance level of the subject is based on neural activity underlying the stimulus anticipation.

8. The method according to claim 7, wherein adapting the cognitive task based on the neural performance level comprises adapting an aspect of the cognitive task relating to stimulus anticipation.

9. The method according to any one of claims 1 to 8, wherein the cognitive task requires the subject to respond to the stimulus.

10. The method according to claim 9, wherein the one or more stimulus-related events comprises response preparation.

11. The method according to claim 10, wherein determining a neural performance level of the subject is based on neural activity underlying the response preparation.

12. The method according to claim 11, wherein adapting the cognitive task based on the neural performance level comprises adapting an aspect of the cognitive task relating to response preparation.

13. The method according to any one of claims 1 to 12, wherein the cognitive task targets an aspect of cognition selected from the group consisting of: attention, working memory, task-switching, goal management, target search, target discrimination, and any combination thereof.

14. The method according to claim 13, wherein the cognitive task is an attention task.

15. The method according to claim 14, wherein the attention task is a selective attention task.

16. The method according to claim 15, wherein the selective attention task requires the subject to discriminate target information from distractions.

17. The method according to any one of claims 1 to 16, wherein the stimulus or sequence of stimuli comprises a visual stimulus, an auditory stimulus, a tactile stimulus, an olfactory stimulus, or any combination thereof.

18. The method according to any one of claims 1 to 17, wherein the monitoring comprises measuring neural activity of the subject by electroencephalography (EEG), functional magnetic resonance imaging (fMRI), near-infrared spectroscopy (NIRS), electrocortocography (ECoG), or a combination thereof, as the subject performs the cognitive task.

19. The method according to claim 18, wherein the monitoring comprises co-registering the neural activity of the subject with a 3-dimensional (3D) structural model of the subject's brain.

20. The method according to claim 19, comprising producing the 3D model of the subject's brain by performing a magnetic resonance imaging (MRI) structural brain scan on the subject prior to or during the presenting of the cognitive task.

21. The method according to any one of claims 1 to 20, comprising providing an indication to the subject of the subject's neural performance level.

22. The method according to claim 21, wherein the indication comprises an award.

23. The method according to any one of claims 1 to 22, wherein the subject has a cognitive deficit selected from the group consisting of: attention deficit hyperactivity disorder (ADHD), post-traumatic stress disorder (PTSD), major depressive disorder, dementia, or a combination thereof.

24. A system for neural activity detection and adaptive training, the system comprising:
a user interface;
a neural activity detector;
a computing device comprising a non-transitory computer readable medium storing instructions that, when executed, cause the computing device to:
present, through the user interface, a first cognitive task to a subject comprising a stimulus or sequence of stimuli to generate stimulus-related events in the brain of the subject;
receive electrical signals from the neural activity detector during the presentation of the cognitive task that represents neural activity underlying the stimulus-related events in the brain of the subject;
map the electrical signals in real-time onto a 3D model of the subject's brain to locate the neural activity;
measure the strength of the located neural activity;
determine a neural performance level of the subject based on the measured neural activity;
present, through the user interface, a second cognitive task to the subject adapted according to the determined neural performance level.

25. The system according to claim 24, wherein the user interface comprises a display device adapted to relay a visual stimulus of the first and second cognitive tasks to the subject.

26. The system according to claim 24 or 25, wherein the user interface comprises an auditory device adapted to relay an audible stimulus of the first and second cognitive tasks to the subject.

27. The system according to any one of claims 24 to 26, wherein the user interface comprises a tactile stimulator adapted to relay a tactile stimulus of the first and second cognitive tasks to the subject.

28. The system according to any one of claims 24 to 28, wherein the user interface comprises an olfactory stimulator adapted to relay an olfactory stimulus of the first and second cognitive tasks to the subject.

29. The system according to any one of claims 24 to 28, wherein the user interface comprises a taste stimulator adapted to relay a taste stimulus of the first and second cognitive tasks to the subject.

30. The system according to any one of claims 24 to 29, wherein the neural activity detector comprises a device selected from the group consisting of: an electroencephalogram (EEG) device, a functional magnetic resonance imaging (fMRI) device, a near-infrared spectroscopy (NIRS) device, an electrocortocography (ECoG) device, and a combination thereof.

31. The system according to any one of claims 24 to 30, wherein the 3D model of the subject's brain is generated from a magnetic resonance imaging (MRI) structural brain scan of the subject's brain.

32. The system according to claim 31, wherein the system further comprises a MRI scanner and the non-transitory computer readable medium further stores instructions that, when executed, cause the computing device to trigger the MRI scanner to generate the MRI structural brain scan of the subject's brain.

33. The system according to any one of claims 24 to 32, wherein the non-transitory computer readable medium further stores instructions that, when executed, cause the computing device to trigger the user interface to provide feedback to the subject based on the neural performance level of the subject.

34. The system according to any one of claims 24 to 33, wherein the user interface further comprises a user input device adapted to allow the subject to input a behavioral response to the stimulus or sequence of stimuli.

35. The system according to any one of claims 24 to 34, wherein the non-transitory computer readable medium further stores instructions that, when executed, cause the computing device to assess the subject's behavioral performance level on the cognitive tasks and adapt the cognitive task based on both the neural performance level and the behavioral performance level.

36. The system according to any one of claims 24 to 35, wherein the non-transitory computer readable medium further stores instructions that, when executed, cause the computing device to trigger the user interface to provide feedback to the subject based on the behavioral performance level of the subject.
